(19) United States
(12) Patent Application Publication
(10) Pub. No.: US 2026/0317422 A1
(43) Pub. Date: Oct. 15, 2026

(54) A COMBINATION COMPRISING LAG-3 TARGETING MOIETY AND INTERLEUKIN-10

(71) Applicant: Elixiron Immunotherapeutics (Hong Kong) Limited, Central Hong Kong (CN)

(72) Inventors: Hung-Kai Chen, Los Altos, CA (US); Tsung-Hao Chang, New Taipei City (TW); Wei Huang, Taipei City (TW); Shih-Rang Yang, New Taipei City (TW); Yi-Ting Kuo, Taichung City (TW); Jing-Yi Huang, Taipei City (TW); Po-Hao CHANG, New Taipei City (TW)

(21) Appl. No.: 19/483,287

(22) PCT Filed: May 10, 2024
(86) PCT No.: PCT/US2024/028700

§ 371 Date: Nov. 11, 2025

Related U.S. Application Data
(60) Provisional application No. 63/501,463, filed on May 11, 2023.

(51) Int. Cl. C07K 16/28; A61K 38/00; A61P 35/00; C07K 14/54

(52) U.S. Cl. CPC C07K 16/2803; A61P 35/00; C07K 14/5428; A61K 38/00

(57) ABSTRACT

The present disclosure provides a combination comprising LAG-3 binding moiety and IL-10. Said combination benefits the cancer treatment.

### CROSS-REFERENCE TO RELATED APPLICATION

This application claims the benefit of U.S. Provisional Application No. 63/501,463, filed May 11, 2023, the entire contents of which are incorporated herein by reference for all purposes.

### SEQUENCE LISTING

The contents of the electronic sequence listing filed herewith, titled “24P0266-Seqlist-F.xml”, created May 10, 2024, having a file size of 70,195 bytes, is hereby incorporated by reference in its entirety.

### FIELD OF THE INVENTION

The present disclosure relates to a combination comprising a LAG-3 binding moiety and interleukin-10 (IL-10), which provides a beneficial effect in the treatment of cancer. Said combination comprising a LAG-3 binding moiety and IL-10 provides an increased anti-cancer activity.

### BACKGROUND OF THE INVENTION

IL-10 is typically considered to be an anti-inflammatory cytokine because of its multiple functions to regulate antigen presenting cells, CD4 T cells and other immune cells. However, administration of IL-10 also triggers strong anti-tumor responses in cancer clinical trials as well as in various mouse tumor models. The anti-tumor role of IL-10 is uncovered specifically in CD8 T cells. IL-10 could directly induce polyclonal CD8 T cell expansion after TCR-mediated T cell activation¹. In a tumor-peptide vaccination mouse model, administration of IL-10 induced more memory-type CD8 T cells and resulted in better tumor rejection². Administration of IL-10 directly activated and expanded tumor-resident CD8 T cells in mouse tumors³. IL-10 was also able to reverse the exhaustion of CD8 T cells through reprogramming of their metabolism⁴.

A series of clinical trials for the treatment of solid tumors have been performed with PEGylated-IL-10 (Pegilodecakin)⁵. PEGylated-IL-10 induced activation and proliferation of intratumoral CD8 T cells in cancer patients⁵. However, Pegilodecakin treatment dosage and regimen are limited by the observed adverse effects. Thus, there is a need for novel IL-10 therapies with improved therapeutic efficacy and safety profiles.

Immune checkpoint inhibitor molecules such as PD-1 and LAG-3 are highly expressed on tumor-infiltrated CD8 T cells, and checkpoint inhibitor-blocking antibodies have demonstrated significant antitumor activity and clinical benefits in several cancer indications, albeit LAG-3 blocking has so far shown significant clinical efficacy only when combined with PD-1 blocking therapeutics. Despite this success, most patients still do not see any long-term benefit from current checkpoint inhibitor therapeutics.

Many anti-LAG-3 monoclonal antibodies including Relatlimab (Bristol-Myers Squibb), Favezelimab (Merck) and Ieramilimab (Novartis), are under investigation in clinical trials in combination mainly with additional immune checkpoint inhibitors. Due to its limited efficacy as single agent and not clear biological function, the feasibility of the combination between LAG3 inhibitors and other immunotherapies needs investigation. Therefore, there is a great need for additional agents and novel combinatorial therapy approaches to enhance response rates and broaden the potential of immunotherapy to a greater number of cancer patients.

### SUMMARY OF THE INVENTION

The present invention provides a combination of LAG-3 targeting moiety and IL-10. The combination described herein can provide beneficial effects in the treatment of cancer. Provided herein is also a new IL-10 fusion protein comprising a LAG-3-targeting moiety linked to IL-10. In one embodiments, the LAG-3-targeting moiety-IL-10 fusion protein shows superior CD8 stimulating activity as compared to a similar IL-10 fusion protein targeting PD-1. In another embodiments, the fusion molecule is administered as a single agent and achieves a better therapeutic effect compared to a combination using equal dose of monotherapy.

The presented disclosure provides an anti-LAG-3-IL-10 fusion protein with superior CD8 activation effects comparing to anti-PD-1-IL-10 fusion proteins and to the combination of LAG-3 antibody and IL-10.

In a first aspect, there is provided a combination of a LAG-3-targeting moiety, preferably an anti-LAG-3 antibody or a fragment thereof, and an IL-10, preferably an IL-10 monomer or dimer.

In one embodiment, the LAG-3 targeting moiety may be fused to the IL-10 via a linker; optionally, the IL-10 may be fused to N-terminal or C-terminal of the LAG-3-targeting moiety with or without a linker, e.g. a peptide linker.

In one embodiment, the LAG-3 targeting moiety blocks LAG-3 from binding to ligands. Preferably, the ligands may comprise MHC class II, FGL-1, Gal-3 or lymph node sinusoidal endothelial cell C-type lectin (LSECtin).

In one embodiment, the linker may comprise the amino acid sequence of SEQ ID NO: 11-17. In another embodiment, a fusion of the LAG-3 targeting moiety and IL-10 is selected from the group consisting of Antibody-IL10; scFv-IL10; scFv-IL10-IL10; VH-IL10-IL10-VL; IL-10-scFv-IL-10; ScFv-CH1-IL-10; IL-10-scFv; Fab-IL-10; F(ab′) 2-IL-10 and scFv-Fc-IL-10. In a preferred embodiment, the IL-10 may be fused to the heavy chain of the antibody in the Antibody-IL10.

In one embodiment, the IL-10 may be a naturally-occurring or engineered variant of IL-10 that retains its cytokine activity.

In one embodiment, the IL-10 may be a synthetically modified version of IL-10 that retains its cytokine activity.

In one embodiment, the IL-10 may contain a substitution in certain amino acids as follows (relative to amino acids of SEQ ID NO: 19): (1) R104Q; (2) any one of R107A, R107E, R107Q and R107D, or; (3) a combination thereof. Amino acid substitution combinations may preferably be R104Q/R107A, R104Q/R107E, R104Q/R107Q or R104Q/R107D.

In one embodiment, the IL-10 may comprise an amino acid sequence having at least 70%, 75%, 80%, 85%, 90%, 91%, 92%, 93%, 94%, 95%, 96%, 97%, 98%, or 99% identity with SEQ ID NO: 19-29.

In one embodiment, the IL-10 may be a monomer or a dimer.

In one embodiment, wherein the anti-LAG-3 antibody or a fragment thereof may comprise a heavy chain (HC) fused via a linker to the IL-10 and a light chain (LC), and the HC and the LC may comprise an amino acid sequence, respectively, having at least 70%, 75%, 80%, 85%, 90%, 91%, 92%, 93%, 94%, 95%, 96%, 97%, 98%, or 99% identity to:
(1) SEQ ID NO: 1 and SEQ ID NO: 2;
(2) SEQ ID NO: 3 and SEQ ID NO: 4;
(3) SEQ ID NO: 5 and SEQ ID NO: 6; or
(4) SEQ ID NO: 32 and SEQ ID NO: 33.

In one embodiment, (i) the antibody is a human, humanized or chimeric antibody; (ii) the antibody is a full length antibody of class IgG, optionally, wherein the class IgG antibody has an isotype selected from IgG1, IgG2, IgG3, and IgG4; (iii) the antibody comprises an Fc region variant, optionally an Fc region variant that alters effector function and/or a variant that alters antibody half-life; (iv) the antibody is an antibody fragment, optionally selected from the group consisting of F(ab′)₂, Fab′, Fab, Fv, single domain antibody (VHH), and scFv; or (v) the antibody is a multi-specific antibody, optionally a bispecific antibody.

In one embodiment, the LAG-3 antibody or a fragment thereof may comprise 15011, Relatlimab, Ieramilimab or Favezelimab.

The present disclosure also provides embodiments of polynucleotide or vector encoding the fusion of the IL-10 and the LAG-3 targeting moiety of the above.

In one embodiment, the polynucleotide is an RNA encoding the fusion of the IL-10 and the LAG-3 targeting moiety. In a preferred embodiment, the RNA is formulated or is to be formulated as particles. In one embodiment, the fusion is complexed with lipid nanoparticles (LNP).

In at least one embodiment the present disclosure provides a host cell comprising the polynucleotide or vector of the above; optionally, wherein the host cell is selected from a group consisting of Chinese hamster ovary (CHO) cell, a myeloma cell comprising Y0, NS0 or Sp2/0, a monkey kidney cell comprising COS-7, a human embryonic kidney line comprising 293, a baby hamster kidney cell (BHK), a mouse Sertoli cell comprising TM4, an African green monkey kidney cell comprising VERO-76, a human cervical carcinoma cell (HELA), a canine kidney cell, a human lung cell comprising W138, a human liver cell comprising HepG2, a mouse mammary tumor cell, a TR1 cell, a Medical Research Council 5 (MRC 5) cell, a FS4 cell, neutrophils, eosinophils, basophils, mast cells, monocytes, macrophages, dendritic cells, and lymphocytes including natural killer cells, B cells and T cells.

In at least one embodiment, the present disclosure provides a method of producing a fusion of the IL-10 and the LAG-3 targeting moiety, comprising culturing the host cell of the above.

In at least one embodiment, the present disclosure provides a pharmaceutical composition comprising a combination of the above, and a pharmaceutically acceptable carrier, diluent or excipient.

In at least one embodiment, the present disclosure provides a use of a combination of the above for the manufacture of a medicament for treating cancers.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE FIGURES

FIG. 1 depicts examples of the sequences of anti-LAG3 antibody-IL-10 and anti-PD-1 antibody-IL-10 fusion proteins. Fusion proteins in antibody-IL-10 formats (A-G), Variable domain: UPPERCASE; Constant domain: lower case in bold; Linker: UPPERCASE ITALICS; IL-10: UPPERCASE UNDERLINED. Fusion proteins based on ScFv (H-I), Heavy chain variable domain: UPPERCASE; Light chain variable domain: lower case in bold; Linker: UPPERCASE ITALICS; Fc: lower case UNDERLINED; IL-10: UPPERCASE UNDERLINED; HIS tag: UPPERCASE IN BOLD.

FIG. 2 depicts the enhanced anti-tumor activity of the combination of anti-LAG3 antibody and IL-10 compared to either anti-LAG3 antibody alone or IL10 alone. Mice bearing established CT26 tumor (50-100 mm³) were divided into six groups, each receiving a different treatment (PBS, IL-10 R104Q/R107A (IL10 (QA))-Fc, 15011, 15011 plus IL10 (QA)-Fc, Clone 13, and Clone 13 plus IL10 (QA)-Fc. n=8). Tumor volume is represented as the mean.

FIG. 3(A) depicts CD8 stimulating activity of LAG-3-targeted IL-10 fusion protein (Relatlimab-IL10), untargeted IL-10 (IL10-Fc) and combination of Relatlimab and IL10-Fc. Data are shown as mean±SD. FIG. 3(B) depicts enhanced CD8 stimulating activity of LAG-3-targeted IL-10 fusion protein (Relatlimab-IL-10) when compared to non-LAG3 targeted IL-10 fusion proteins (anti-CSF-1R-IL10 and IL10-Fc). Data are shown as mean±SD.

FIG. 4 depicts superior CD8 T cell stimulating activity of LAG-3-targeted IL-10 fusion proteins compared to PD-1-targeted IL-10 fusion proteins. Various fusion proteins were purified and added to CD8 assay. Data of granzyme B and IFN-γ secretion by CD8 T cells from two donors were shown in (A) Donor 1 and (B) Donor 2. Data are shown as mean±SD.

FIG. 5 depicts dose-dependent CD8 activation activity of various anti-LAG3 antibody-IL-10 fusion proteins in culture supernatant secreted by HeLa cells transfected with increasing amounts of plasmid. Data are shown as mean±SD.

FIG. 6(A) depicts anti-tumor activity of anti-LAG3 antibody-IL-10 mRNA in MC38 tumor model. Mice bearing established MC38 tumor (50-100 mm³) were divided into two treatment groups (Luciferase mRNA and anti-LAG-3 15011-IL10 mRNA). Intratumoral injection of mRNA in LNP was performed on day 9, 12, 16 and 19. Tumor volume is represented as the mean±SEM. FIG. 6(B) depicts the expression of 15011-IL10 protein in mRNA-LNP injected tumor. Protein level of 15011-IL10 in tumor lysate was determined by ELISA.

FIG. 7 depicts the enhanced anti-tumor activity of anti-LAG3 antibody-IL-10 fusion compared to combination of anti-LAG3 antibody and IL-10 in CT26 tumor model. Mice bearing established CT26 tumor (50-100 mm³) were divided into three mRNA-LNP treatment groups (non-coding mRNA control, 15011 antibody mRNA plus IL-10 mRNA, and 15011-IL10 fusion protein mRNA). Intratumoral injection of mRNA in LNP was performed on day 10, and 17. Tumor volume is represented as the mean.

FIG. 8 depicts examples of various fusion formats of LAG3-targeted IL-10 fusion protein. (1) Antibody-IL10: consisting of two heavy chain-IL10 fusion (HC-IL10) and two light chains. HC-IL10: an IL-10 monomer is fused to the C-terminus of heavy chain. (2) scFv-IL10: scFv is fused to the N-terminus of one IL-10 monomer. (3) scFv-IL10-IL10: scFv is fused to the N-terminus of first IL-10 monomer, which is fused to the N-terminus of a second IL-10 monomer. (4) VH-IL10-IL10-VL: VH (heavy chain variable domain) is fused to the N-terminus of two sequential IL-10 monomer and to the N-terminus of VL (light chain variable domain). (5) IL10-scFv-IL10: the first IL10 monomer is fused to the N-terminal end of the scFv and the second IL10 is fused to the C-terminal end of the scFv. (6) scFv-CH1-IL-10: scFv is fused to the N-terminus of CH1, which is fused to the N-terminus of one IL-10 monomer. (7) IL-10-scFv: one IL-10 monomer is fused to the N-terminus of scFv. (8) Fab-IL10: one VH-CH1-IL10 fusion and one light chain-IL10 fusion. VH-CH1-IL10: one IL-10 monomer is fused to the C-terminus of VH-CH1. Light chain-IL10: one IL-10 monomer is fused to the C-terminus of light chain (VL-CL-IL10). (9) F(ab′) 2-IL10: two VH-CH1-hinge-IL10 fusion and two light chains. VH-CH1-hinge-IL10 fusion: one IL-10 monomer is fused to the C-terminus of hinge of Fab heavy chain (VH1-CH1). (10) scFv-Fc-IL10: scFv is fused to the N-terminus of the human IgG hinge-CH2/CH3 domain to an IL-10 monomer.

FIG. 9(A) depicts Western blotting analysis of expression of ten different fusion formats of anti-LAG3 antibody-IL10 fusion in HeLa culture supernatant. HeLa cells were transfected with plasmids encoding different fusion proteins. Culture supernatants were collected 72 hours later and were subjected to Western blot under reducing condition to detect fusion protein fragment containing IL-10 using anti-IL10 antibody. Lane 1: antibody-IL10; Lane 2: scFv-IL10; Lane 3: scFv-IL10-IL10; Lane 4: VH-IL10-IL10-VL; Lane 5: IL10-scfv-IL10; Lane 6: scFv-CH1-IL10; Lane 7: IL10-scFv; Lane 8: Fab-IL10; Lane 9: F(ab′)₂-IL10; Lane 10: scFv-Fc-IL10; Lane M: molecular weight markers. (kDa). FIG. 9(B) depicts the fold change of IFN-γ expression in CD8 activation assay adding culture supernatants from HeLa cells transduced with various anti-LAG3 antibody-IL10 fusion constructs and control constructs. Construct 1: Antibody-IL10; 2: scFv-IL10; 3: scFv-IL10-IL10; 4: VH-IL10-IL10-VL; 5: IL10-scfv-IL10; 6: scFv-CH1-IL10; 7: IL10-scFv; 8: Fab-IL10; 9: F (ab) 2-IL10; 10: scFv-Fc-IL10; 11: IL10; 12: scFv. Data are shown as mean±SEM of four independent experiments.

FIG. 10(A) depicts Western blotting analysis of expression of three formats of anti-LAG3 antibody-IL10 fusion proteins including 15011 antibody-IL10, 15011 scFv-IL10-IL10 and 15011 scFv-Fc-IL10. IL-10 containing fragment of fusion proteins in HeLa culture supernatants were collected after transfection and were subjected to Western blot under reducing condition to detect fusion protein fragment containing IL-10 using anti-IL10 antibody. Lane 1:15011 antibody-IL10; Lane 2:15011 scFv-IL10-IL10; Lane 3:15011 scFv-Fc-IL10; Lane 4: empty vector control. FIG. 10(B) depicts the CD8 activation activity of culture supernatants from HeLa cells transduced with increasing amount of plasmids encoding 15011 antibody, 15011 antibody-IL10, 15011 scFv-IL10-IL10, 15011 scFv-Fc-IL10 and IL10. Granzyme B secretion (mean±SD) was determined by ELISA.

### DETAILED DESCRIPTION

### Definitions

Generally, the nomenclature used herein and the techniques and procedures described herein include those that are well understood and commonly employed by those of ordinary skill in the art, such as the common techniques and methodologies described in Sambrook et al., Molecular Cloning-A Laboratory Manual (2nd Ed.), Vols. 1-3, Cold Spring Harbor Laboratory, Cold Spring Harbor, N.Y., 1989 (hereinafter “Sambrook”); Current Protocols in Molecular Biology, F. M. Ausubel et al., eds., Current Protocols, a joint venture between Greene Publishing Associates, Inc. and John Wiley & Sons, Inc. (supplemented through 2011) (hereinafter “Ausubel”); Antibody Engineering, Vols. 1 and 2, R. Kontermann and S. Dubel, eds., Springer-Verlag, Berlin and Heidelberg (2010); Monoclonal Antibodies: Methods and Protocols, V. Ossipow and N. Fischer, eds., 2nd Ed., Humana Press (2014); Therapeutic Antibodies: From Bench to Clinic, Z. An, ed., J. Wiley & Sons, Hoboken, N.J. (2009); and Phage Display, Tim Clackson and Henry B. Lowman, eds., Oxford University Press, United Kingdom (2004).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for the purpose of describing particular embodiments only and is not intended to be limiting. For purposes of interpreting this disclosure, the following description of terms will apply and, where appropriate, a term used in the singular form will also include the plural form and vice versa.

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50,” includes “2 to 25,” “5 to 20,” “25 to 50,” “1 to 10,” etc.

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. It should be understood that, operably linked” elements may be contiguous or non-contiguous. In the context of a polypeptide, “operably linked” refers to a physical linkage (e.g., directly or indirectly linked) between amino acid sequences (e.g., different domains) to provide for a described activity of the polypeptide. In the present disclosure, various domains of the recombinant polypeptides of the disclosure may be operably linked to retain proper folding, processing, targeting, expression, binding, and other functional properties of the recombinant polypeptides in the cell. Operably linked domains of the recombinant polypeptides of the disclosure may be contiguous or non-contiguous (e.g., linked to one another through a linker).

The term “polynucleotide” refers to a biopolymer composed of nucleotide monomers covalently bonded in a chain. The examples of polynucleotide comprise DNA (deoxyribonucleic acid) and RNA (ribonucleic acid), e.g. messenger RNA. The polynucleotide may be delivered to the subject in need in a way known to the art so as to express the protein of interest, e.g. fusion proteins thereof, in the subject in need directly.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al, J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “RNA encoding” means that the RNA, if present in the appropriate environment, preferably within a cell, can direct the assembly of amino acids to produce the protein or peptide is encodes during the process of translation. Preferably, RNA according to the invention is able to interact with the cellular translation machinery allowing translation of the protein or peptide.

The term “pharmaceutically acceptable carrier, diluent or excipient” as used herein refers to any suitable substance that provides a pharmaceutically acceptable carrier, additive or diluent for administration of a compound(s) of interest to a subject. As such, “pharmaceutically acceptable carrier, diluent or excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “recombinant” or “engineered” nucleic acid molecule or polypeptide as used herein, refers to a nucleic acid molecule or polypeptide that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule can be one which: 1) has been synthesized or modified in vitro, for example, using chemical or enzymatic techniques; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. Another non-limiting example of a recombinant nucleic acid and recombinant protein is an IL-10 polypeptide variant as disclosed herein.

“IL10” or “IL-10,” as used herein, refers to the cytokine, interleukin 10, also known as cytokine synthesis inhibitory factor (CSIF), and is intended to also include naturally-occurring variants, engineered variants, and/or synthetically modified versions of interleukin 10 that retain its cytokine functions. Amino acid sequences of various exemplary IL-10 polypeptides and recombinant IL-10 fusion constructs are provided in Table 2 below and the attached Sequence Listing. Other exemplary engineered and/or modified IL-10 polypeptides that retain cytokine functions are known in the art (see e.g., U.S. Pat. No. 7,749,490 B2; US 2017/0015747 A1; Naing, A. et al. “PEGylated IL-10 (Pegilodecakin) Induces Systemic Immune Activation, CD8+ T Cell Invigoration and Polyclonal T Cell Expansion in Cancer Patients.” Cancer Cell 34, 775-791.e3 (2018); Gorby, C. et al. “Engineered IL-10 variants elicit potent immunomodulatory effects at low ligand doses.” Sci Signal 13, (2020); Yoon, S. I. et al. “Epstein-Barr virus IL-10 engages IL-10R1 by a two-step mechanism leading to altered signaling properties.” J Biol Chem 287, 26586-26595 (2012)).

“Fusion protein,” as used herein, refers to two or more protein and/or polypeptide molecules that are linked (or “fused”) in a configuration that does not occur naturally. An exemplary fusion protein of the present disclosure includes the “IL-10-Fc” fusion protein that comprises an IL-10 polypeptide covalently linked through a polypeptide linker sequence at its C-terminus to an immunoglobulin Fc region polypeptide. Fusion proteins of the present disclosure also include “antibody fusions” that comprise a full-length IgG antibody (with both a heavy chain and a light chain polypeptide) that is covalently linked through a polypeptide linker sequence at its heavy chain C-terminus to an IL-10 polypeptide. As used herein, the terms “linked,” “fused”, or “fusion” are used interchangeably. These terms refer to the joining together of two or more elements or components or domains.

“Polypeptide linker” or “linker sequence” as used herein refers to a chain of two or more amino acids with each end of the chain covalently attached to a different polypeptide molecule, thereby functioning to conjugate or fuse the different polypeptides. Typically, polypeptide linkers comprise polypeptide chains of 1 to 42 amino acids, preferably 5 to 30 amino acids. A wide range of polypeptide linkers are known in the art and can be used in the compositions and methods of the present disclosure. Exemplary polypeptide linkers include those shown in Table 1, and other specific linker sequences as disclosed elsewhere herein.

TABLE 1
Exemplary polypeptide linkers

| | | |
|---|---|---|
| Linker 1 | (GGGGS)n; n = 1-6 | SEQ ID NO: 11 |
| | repeat(s) | |
| Linker 2 | (SSSSG)n; n = 1-6 | SEQ ID NO: 12 |
| | repeat(s) | |
| Linker 3 | (G)n; n = 1-6 | SEQ ID NO: 13 |
| | repeat(s) | |
| Linker 4 | (EAAAK)n; n = 1-6 | SEQ ID NO: 14 |
| | repeat(s) | |
| Linker 5 | (XP)n; n = 1-6 | SEQ ID NO: 15 |
| | repeat(s) | |
| Linker 6 | ENLYFQ(-G/S) | SEQ ID NO: 16 |
| Linker 7 | LGGGGSGGGGSGGGG | SEQ ID NO: 17 |

“Antibody,” as used herein, refers to a molecule comprising one or more polypeptide chains that specifically binds to, or is immunologically reactive with, a particular antigen. Exemplary antibodies of the present disclosure include monoclonal antibodies, polyclonal antibodies, chimeric antibodies, humanized antibodies, human antibodies, antibody fusions, multispecific antibodies (e.g., bispecific antibodies), monovalent antibodies (e.g., single-arm antibodies), multivalent antibodies, antigen-binding fragments (e.g., Fab′, F(ab′)₂, Fab, Fv, rIgG, and scFv fragments), and synthetic antibodies (or antibody mimetics).

“Full-length antibody,” “intact antibody,” or “whole antibody” are used herein interchangeably to refer to an antibody having a structure substantially similar to a native antibody structure or having heavy chains that contain an Fc region as defined herein.

A “fragment” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments or portions of a full-length antibody. Examples of binding fragments encompassed within the term “antigen-binding portion” or “antigen-binding fragment” of an antibody, e.g., an anti-LAG-3 antibody described herein, include:
(1) a Fab fragment (fragment from papain cleavage) or a similar monovalent fragment consisting of the VL, VH, LC and CH1 domains;
(2) a F(ab′)₂ fragment (fragment from pepsin cleavage) or a similar bivalent fragment comprising two Fab fragments linked by a disulfide bridge at the hinge region;
(3) a Fd fragment consisting of the VH and CH1 domains;
(4) a Fv fragment consisting of the VL and VH domains of a single arm of an antibody,
(5) a single domain antibody (dAb) fragment (Ward et al., (1989) Nature 341:544-46), which consists of a VH domain;
(6) a bi-single domain antibody which consists of two VH domains linked by a hinge (dual-affinity re-targeting antibodies (DARTs));
(7) a dual variable domain immunoglobulin;
(8) an isolated complementarity determining region (CDR); and
(9) a combination of two or more isolated CDRs, which can optionally be joined by a synthetic linker.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. In some embodiments, an antibody is an antigen-binding fragment.

“Fc region” refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain may vary, the human IgG heavy chain Fc sequence is usually defined to stretch from an amino acid residue at about position Cys226, or from about position Pro230, to the carboxyl-terminus of the Fc sequence. However, the C-terminal lysine (Lys447) of the Fc sequence may or may not be present. The Fc sequence of an immunoglobulin generally comprises two constant domains, a CH2 domain and a CH3 domain, and optionally comprises a CH4 domain.

“Antibody fusion” refers to an antibody that is covalently conjugated (or fused) to a polypeptide or protein, typically via a linker to a terminus of the antibody's light chain (LC) or heavy chain (HC). Exemplary antibody fusions of the present disclosure include an anti-LAG-3 antibody fused to a recombinant IL-10 polypeptide via a 15 amino acid polypeptide linker (e.g., SEQ ID NO: 1) from the C-terminus of the antibody heavy chain to the N-terminus of the IL-10 polypeptide. Antibody fusions are labeled herein with an “antibody-polypeptide” nomenclature to indicate the fusion components, such as “Ab-IL-10” or “anti-LAG3 antibody-IL-10.” As described elsewhere herein, an antibody fusion of the present disclosure can include a full-length IgG antibody, comprising a dimeric complex of heavy chain-light chain pairs, where each heavy chain C-terminus is linked through a polypeptide linker sequence to an IL-10 polypeptide.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

The term “LAG-3” refers to Lymphocyte Activation Gene-3. The term “LAG-3” includes variants, isoforms, homologs, orthologs and paralogs. For example, antibodies specific for a human LAG-3 protein may, in certain cases, cross-react with a LAG-3 protein from a species other than human. In other embodiments, the antibodies specific for a human LAG-3 protein may be completely specific for the human LAG-3 protein and may not exhibit species or other types of cross-reactivity, or may cross-react with LAG-3 from certain other species, but not all other species (e.g., cross-react with monkey LAG-3 but not mouse LAG-3). The term “human LAG-3” refers to human sequence LAG-3, such as the complete amino acid sequence of human LAG-3 having GenBank Accession No. NP_002277. The term “mouse LAG-3” refers to mouse sequence LAG-3, such as the complete amino acid sequence of mouse LAG-3 having GenBank Accession No. NP 032505. LAG-3 is also known in the art as, for example, CD223. The human LAG-3 sequence may differ from human LAG-3 of GenBank Accession No. NP_002277 by having, e.g., conserved mutations or mutations in non-conserved regions and the LAG-3 has substantially the same biological function as the human LAG-3 of GenBank Accession No. NP_002277. For example, a biological function of human LAG-3 is having an epitope in the extracellular domain of LAG-3 that is specifically bound by an antibody of the instant disclosure or a biological function of human LAG-3 is binding to MHC Class II molecules, FGL-1, Gal-3 or lymph node sinusoidal endothelial cell C-type lectin (LSECtin).

As used herein, the terms “combination therapy” and “therapeutic combination” refer to treatments in which at least one LAG-3 targeting moiety, e.g. anti-LAG-3 antibody or a fragment thereof, and IL-10, and optionally additional therapeutic agents, each are administered to a patient in a coordinated manner, over an overlapping period of time. The period of treatment with the IL-10 (the “IL-10 treatment”) is the period of time that a patient undergoes treatment with the IL-10; that is, the period of time from the initial dosing with the IL-10 through the final day of a treatment cycle. Similarly, the period of treatment with the at least one LAG-3 targeting moiety (the “anti-LAG3 treatment”) is the period of time that a patient undergoes treatment with the LAG-3 targeting moiety; that is, the period of time from the initial dosing with the LAG-3 targeting moiety through the final day of a treatment cycle. In the methods and therapeutic combinations described herein, the IL-10 treatment overlaps by at least one day with the anti-LAG3 treatment. In certain embodiments, the IL-10 treatment and the anti-LAG3 treatment are the same period of time. In some embodiments, the IL-10 treatment begins prior to the anti-LAG3 treatment. In other embodiments, the IL-10 treatment begins after the anti-LAG3 treatment. In certain embodiments, the IL-10 treatment is terminated prior to termination of the anti-LAG3 treatment. In other embodiments, the IL-10 treatment is terminated after termination of the anti-LAG3 treatment.

A “cancer” refers a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include liver cancer, bone cancer, pancreatic cancer, skin cancer, oral cancer, cancer of the head or neck, breast cancer, lung cancer, including small cell and non-small cell lung cancer, cutaneous or intraocular malignant melanoma, renal cancer, uterine cancer, ovarian cancer, colorectal cancer, colon cancer, rectal cancer, cancer of the anal region, gastric cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, cancers of the childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combination thereof. In another embodiment, the malignant tumor is a gastric cancer or gastroesophageal junction cancer. In another embodiment, the gastric cancer is an adenocarcinoma, lymphoma, gastrointestinal stromal tumor, or carcinoid tumor. In another embodiment, the malignant tumor is chosen from melanoma, non-small cell lung cancer (NSCLC), human papilloma virus (HPV)-related tumor, bladder cancer, head and neck squamous cell carcinoma, renal cell cancer, and gastric adenocarcinoma.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Detailed Description of Various Embodiments

### I. Combination of LAG-3 Targeting Moiety and IL-10

An aspect of the invention provides a combination of LAG-3 targeting moiety and IL-10.

Preferably, the LAG-3 targeting moiety blocks LAG-3 from binding to ligands, including MHC class II, FGL-1, Gal-3 or lymph node sinusoidal endothelial cell C-type lectin (LSECtin). Preferably, the LAG-3 targeting moiety is an anti-LAG-3 antibody or a fragment thereof; and more preferably, the LAG-3 targeting moiety is a neutralizing anti-LAG-3 antibody or a fragment thereof. A further aspect of the disclosure provides a kit comprising an LAG-3 targeting moiety and an IL-10.

In one embodiment, the LAG-3 targeting moiety is an anti-LAG-3 antibody or a fragment thereof. In another embodiment, the anti-LAG-3 antibody is a full-length antibody. In another embodiment, the antibody is a monoclonal, human, humanized, chimeric, or multispecific antibody. In another embodiment, the multispecific antibody is a dual-affinity re-targeting antibody (DART), a DVD-Ig, or bispecific antibody. In another embodiment, the antibody is a F(ab′)2 fragment, a Fab′ fragment, a Fab fragment, a Fv fragment, a scFv fragment, a dsFv fragment, a dAb fragment, or a single chain binding polypeptide.

In various embodiments, the anti-LAG-3 antibody or a fragment thereof is selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the VL and VH regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-LAG-3 antibody or a fragment thereof disclosed herein will have an amino acid sequence having at least 75% amino acid sequence identity with the amino acid sequence of a reference antibody or antigen binding fragment (e.g., heavy chain, light chain, VH, VL, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity.

In some embodiments, the anti-LAG-3 antibody or a fragment thereof is a human antibody. In other embodiments, the anti-LAG-3 antibody or a fragment thereof is a humanized antibody.

In some embodiments, the heavy chain of the anti-LAG-3 antibody or a fragment thereof has a human IgG1 backbone. In other embodiments, the heavy chain of the anti-LAG-3 antibody or a fragment thereof has a human IgG2 backbone. In yet other embodiments, the heavy chain of the anti-LAG-3 antibody or a fragment thereof has a human IgG3 backbone. In still other embodiments, the heavy chain of the anti-LAG-3 antibody or a fragment thereof has a human IgG4 backbone.

In certain embodiments, the anti-LAG-3 antibody is selected from the group consisting of Relatlimab, Ieramilimab, Favezelimab and 15011. In one embodiment, the anti-LAG-3 antibody is Relatlimab. In another embodiment, the anti-LAG-3 antibody is Ieramilimab. In yet another embodiment, the anti-LAG-3 antibody is Favezelimab. In still another embodiment, the anti-LAG-3 antibody is 15011.

It will be apparent to the skilled person that based on the foregoing, pharmaceutical compositions for the treatment of a disease (as specified in more detail below) are also disclosed herein by using the LAG-3 targeting moiety and IL-10 set forth above, as well as methods of treating disease (as specified in more detail below) by making use of such pharmaceutical compositions or combination of the disclosure.

In one embodiment, the LAG-3 targeting moiety and the IL-10 are formulated for intravenous administration. In another embodiment, the LAG-3 targeting moiety and the IL-10 are formulated together. In another embodiment, the LAG-3 targeting moiety and the IL-10 are formulated separately.

When the IL-10 and the LAG-3 targeting moiety of the disclosure may be administered simultaneously via the same route of administration, they can be administered as different formulations or pharmaceutical compositions or as part of a formulation or combined pharmaceutical composition. Also, when the IL-10 and the LAG-3 targeting moiety of the disclosure are to be used as part of a combination treatment regimen, each of the IL-10 and the LAG-3 targeting moiety can be administered according to the same regimen as used when one of the IL-10 and the LAG-3 targeting moiety is used alone. However, when the combined use of the IL-10 and the LAG-3 targeting moiety leads to a synergistic effect, it may also be possible to reduce the amount of the IL-10 and the LAG-3 targeting moiety, while still achieving the desired therapeutic action. This can be useful, for example, to avoid, limit or reduce any unwanted side effects that are associated with the use of the IL-10 and the LAG-3 targeting moiety when used in their usual amounts, while still obtaining the desired pharmacological or therapeutic effect.

The human IL-10 cytokine is a homodimeric protein of two polypeptide subunits. IL-10 signals through an IL-10R consisting of two IL-10 receptor-1 (IL-10Rα subunit) and two IL-10 receptor-2 (IL-10Rβ subunit) proteins. Consequently, the functional receptor consists of four IL-10 receptor molecules. Binding of IL-10 to IL-10R induces STAT3 signaling via the phosphorylation of the cytoplasmic tails of IL-10 receptor by JAK1 and Tyk2. IL-10 is primarily produced by monocytes and, to a lesser extent, lymphocytes, namely type-II T helper cells (T_{H}2), mast cells, CD4⁺CD25⁺Foxp3⁺ regulatory T cells, and in a certain subset of activated T cells and B cells. Table 2 below provides a summary description of the amino sequences of the human IL-10 polypeptide and a recombinant IL-10-Fc fusion construct used in the Examples of the present disclosure, and their sequence identifiers. The sequences also are included in the accompanying Sequence Listing.

TABLE 2
Recombinant IL-10 polypeptides and polypeptide linkers

| | | SEQ |
|---|---|---|
| Description | Sequence | ID NO: |
| IL-10 | MHSSALLCCLVLLTGVRASPGQGTQSENSCTHFPGNLPNML | 18 |
| precursor | RDLRDAFSRVKTFFQMKDQLDNLLLKESLLEDFKGYLGCQ | |
| | ALSEMIQFYLEEVMPQAENQDPDIKAHVNSLGENLKTLRLR | |
| | LRRCHRFLPCENKSKAVEQVKNAFNKLQEKGIYKAMSEFDI | |
| | FINYIEAYMTMKIRN | |
| IL-10 | SPGQGTQSENSCTHFPGNLPNMLRDLRDAFSRVKTFFQMKD | 19 |
| mature form | QLDNLLLKESLLEDFKGYLGCQALSEMIQFYLEEVMPQAEN | |
| | QDPDIKAHVNSLGENLKTLRLRLRRCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 20 |
| R104Q | QLDNLLLKESLLEDFKGYLGCQALSEMIQFYLEEVMPQAEN | |
| | QDPDIKAHVNSLGENLKTLRLQLRRCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 21 |
| R107A | QLDNLLLKESLLEDFKGYLGCQALSEMIQFYLEEVMPQAEN | |
| | QDPDIKAHVNSLGENLKTLRLRLRACHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 22 |
| R107E | QLDNLLLKESLLEDFKGYLGCQALSEMIQFYLEEVMPQAEN | |
| | QDPDIKAHVNSLGENLKTLRLRLRECHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 23 |
| R107Q | QLDNLLLKESLLEDFKGYLGCQALSEMIQFYLEEVMPQAEN | |
| | QDPDIKAHVNSLGENLKTLRLRLRQCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 24 |
| R107D | QLDNLLLKESLLEDFKGYLGCQALSEMIQFYLEEVMPQAEN | |
| | QDPDIKAHVNSLGENLKTLRLRLRDCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 25 |
| K119S | QLDNLLLKESLLEDFKGYLGCQALSEMIQFYLEEVMPQAEN | |
| | QDPDIKAHVNSLGENLKTLRLRLRRCHRFLPCENKSSAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 26 |
| R104Q/R107A | QLDNLLLKESLLEDFKGYLGCQALSEMIQFYLEEVMPQAEN | |
| | QDPDIKAHVNSLGENLKTLRLQLRACHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 27 |
| R104Q/R107E | QLDNLLLKESLLEDFKGYLGCQALSEMIQFYLEEVMPQAEN | |
| | QDPDIKAHVNSLGENLKTLRLQLRECHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 28 |
| R104Q/R107Q | QLDNLLLKESLLEDFKGYLGCQALSEMIQFYLEEVMPQAEN | |
| | QDPDIKAHVNSLGENLKTLRLQLRQCHRFLPCENKSKAVEQ | |
| | VKNAFNKLQEKGIYKAMSEFDIFINYIEAYMTMKIRN | |
| IL-10 | SPGQGTQSENSCTHFPGNLPNMLRDLRDAFSRVKTFFQMKD | 29 |
| R104Q/R107D | QLDNLLLKESLLEDFKGYLGCQALSEMIQFYLEEVMPQAEN | |
| | QDPDIKAHVNSLGENLKTLRLQLRDCHRFLPCENKSKAVEQ | |
| | VKNAFNKLQEKGIYKAMSEFDIFINYIEAYMTMKIRN | |

The term IL-10 or IL-10 polypeptide refers to wild-type IL-10, whether native or recombinant, and encompasses homologs, orthologs, variants, and fragments thereof, as well as IL-10 polypeptides having, for example, a leader sequence (e.g., a signal peptide). As such, an IL-10 polypeptide includes, but is not limited to, a recombinantly-produced IL-10 polypeptide, synthetically-produced IL-10 polypeptide, and IL-10 polypeptide extracted from cells or tissues. As a non-limiting example of IL-10 polypeptides of the disclosure, an amino acid sequence of mature human IL-10 is depicted in SEQ ID NO: 18. Exemplary IL-10 homologs and modified forms thereof from other mammalian species include IL-10 polypeptides from rat (accession NP_036986.2; GI 148747382); cow (accession NP_776513.1; GI 41386772); sheep (accession NP_00 1009327.1; GI 57164347); dog (accession ABY86619.1; GI 166244598), and; rabbit (accession AAC23839.1; GI 3242896). Further examples of IL-10 polypeptides suitable for introduction of amino acid substitutions described herein include, but are not limited to, virus-encoded IL-10 homologs, including IL-10 polypeptides from genera Cytomegalovirus, Lymphocryptovirus, Macavirus, Percavirus, Parapoxvirus, Capripoxvirus, and Avipoxvirus. Non-limiting examples of cytomegalovirus IL-10 polypeptides include those from human cytomegalovirus (accession AAR31656 and ACR49217), Green monkey cytomegalovirus (accession AEV80459), rhesus cytomegalovirus (accession AAF59907), baboon cytomegalovirus (accession AAF63436), owl monkey cytomegalovirus (accession AEV80800), and squirrel monkey cytomegalovirus (accession AEV80955). Examples of cytomegalovirus IL-10 polypeptides include those from Epstein-Barr virus (accession CAD53385), Bonobo herpesvirus (accession XP_003804206.1), Rhesus lymphocryptovirus (accession AAK95412), and baboon lymphocryptovirus (accession AAF23949). Additional information regarding viral IL-10 polypeptides and their control of host immune function can be found in, for example, Slobedman B. et al., J. Virol. October 2009, p. 9618-9629; and Ouyang P. et al., J. Gen. Virol. (2014), 95, 245-262.

An amino acid sequence of wild-type human IL-10 precursor polypeptide (e.g., pre-protein with a signal peptide) is depicted in SEQ ID NO: 18, which is a 178 amino acid residue protein with an N-terminal 18 amino acid signal peptide, that can be removed to generate a 160 amino acid mature protein of SEQ ID NO: 19. However, mature proteins are often used to generate recombinant polypeptide constructs. N-terminal variant of IL-10 has been reported (U.S. Pat. No. 5,328,989). Therefore, for the purpose of the present disclosure, all amino acid numbering is based on the mature polypeptide sequence of the IL-10 protein set forth in SEQ ID NO: 18.

In addition to the naturally-occurring human IL-10, a variety of engineered and/or synthetically modified IL-10 polypeptides that retain the cytokine functions of IL-10 are known in the art. The PEGylated IL-10, Pegilodecakin, has been shown to retain the anti-tumor immune surveillance function of naturally-occurring human IL-10. See, Naing, A. et al. “PEGylated IL-10 (Pegilodecakin) Induces Systemic Immune Activation, CD8⁺ T Cell Invigoration and Polyclonal T Cell Expansion in Cancer Patients.” Cancer Cell 34, 775-791. (2018). The engineered IL-10 variant R5A11 has been shown to have higher affinity to IL-10Rβ, exhibit enhanced signaling activities in human CD8⁺ T-cells, and enhances the anti-tumor function of CAR-T cells. See, Gorby, C. et al. “Engineered IL-10 variants elicit potent immunomodulatory effects at low ligand doses.” Sci Signal 13, (2020). The IL-10 from Epstein-Barr virus has weaker binding to the IL-10R1, but retains the immunosuppressive cytokine activities of human IL-10, while having lost the ability to induce immunostimulatory activities with some cells. See, Yoon, S. I. et al. “Epstein-Barr virus IL-10 engages IL-10R1 by a two-step mechanism leading to altered signaling properties.” J Biol Chem 287, 26586-26595 (2012). U.S. Pat. No. 7,749,490B2 and US2017/0015747A1 described engineered IL-10 mutants (e.g., F129S-IL-10) that exhibit less immunostimulatory activity in MC/9 cell proliferation assay. Generally, it is contemplated that any engineered or modified version of IL-10 polypeptide that retains some IL-10 cytokine function can be used in any of the fusion protein of the present disclosure.

### II. Fusion of LAG-3 Targeting Moiety and IL-10

An aspect of the invention, the combination of the LAG-3 targeting moiety, e.g. an anti-LAG-3 antibody or a fragment thereof, and IL-10 include a fusion of the LAG-3 targeting moiety and IL-10. In one embodiment, the IL-10 is human IL-10. In an embodiment, the LAG-3 targeting moiety may be fused to N-terminus or C-terminus of the IL-10 polypeptide. In another embodiment, the LAG-3 targeting moiety may be simultaneously fused to N-terminal and C-terminal of the IL-10 polypeptide. The LAG-3 targeting moiety may be fused to one or more, e.g. one, two, three or four, IL-10 as long as the fusion still achieving the desired therapeutic action.

In one embodiment, a fusion antibody may be made that comprises all or a portion of the LAG-3 targeting moiety linked to the IL-10. In certain embodiments, only the variable domains of the anti-LAG-3 antibody are linked to the IL-10. In certain embodiments, the VH domain of an anti-LAG-3 antibody is linked to a first IL-10, while the VL domain of an anti-LAG-3 antibody is linked to a second IL-10 that associates with the first IL-10 in a manner such that the VH and VL domains can interact with one another to form an antigen-binding site. In another preferred embodiment, the VH domain is separated from the VL domain by a linker such that the VH and VL domains can interact with one another (e.g., single-chain antibodies). The VH-linker-VL antibody is then linked to the IL-10.

In one embodiment, the VH and VL domains of anti-LAG-3 antibody or a fragment thereof are in the format of Fab or scFv for use in the LAG3-targeting purpose.

In one embodiment, the LAG-3 targeting moiety is an IgG antibody, wherein the fusion protein comprises two heavy chain polypeptides and two light chain polypeptides. In another embodiment, IL-10 monomer is fused at its N-terminus to the C-terminus of heavy chain through a peptide linker.

In one embodiment, the LAG3 targeting moiety is an F(ab′) 2-IL-10 fusion protein. The F(ab′) 2 consists of two heavy chains and two light chains. Each heavy chain is composed of VH, CH1 and a hinge region fused to the N-terminus of one IL-10 monomer. Each light chain is composed of VL and CL.

In some embodiment, the LAG-3 targeting moiety is a scFv. The scFv and the IL-10 is arranged, from amino terminus to carboxyl terminus, as: (i) scFv-IL10; (ii) scFv-IL10-IL10; (iii) IL-10-scFv-IL-10; (iv) IL-10-scFv; (v) scFv-CH1-IL-10. In one embodiment, two IL-10 monomer is inserted between VH and VL of the scFv.

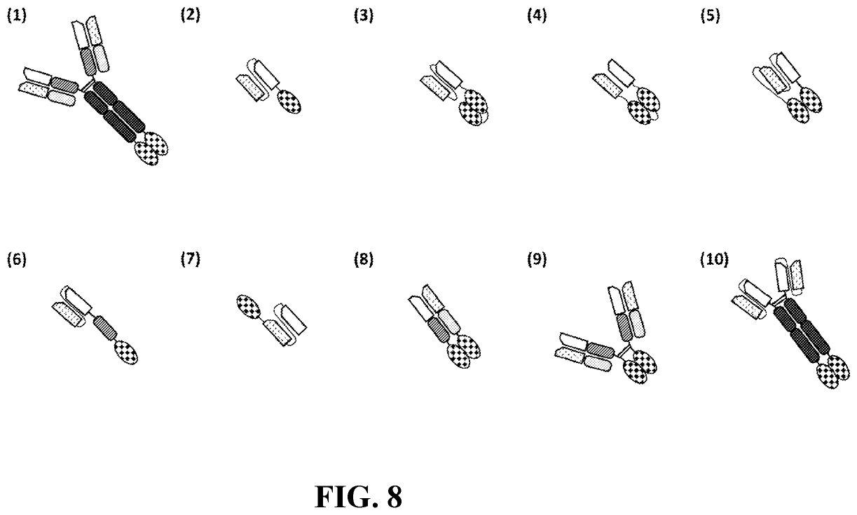

Various fusion constructs of anti-LAG-3-IL-10 fusion protein were shown in FIG. 8. (1) Antibody-IL10: consisting of two heavy chain-IL10 fusion (HC-IL10) and two light chains. HC-IL10: an IL-10 monomer is fused to the C-terminus of heavy chain. (2) scFv-IL10: scFv is fused to the N-terminus of one IL-10 monomer. (3) scFv-IL10-IL10: scFv is fused to the N-terminus of first IL-10 monomer, which is fused to the N-terminus of a second IL-10 monomer. (4) VH-IL10-IL10-VL: VH (heavy chain variable domain) is fused to the N-terminus of two sequential IL-10 monomer and to the N-terminus of VL (light chain variable domain). (5) IL10-scFv-IL10: the first IL10 monomer is fused to the N-terminal end of the scFv and the second IL10 is fused to the C-terminal end of the scFv. (6) scFv-CH1-IL10: scFv is fused to the N-terminus of CH1, which is fused to the N-terminus of one IL-10 monomer. (7) IL-10-scFv: one IL-10 monomer is fused to the N-terminus of scFv. (8) Fab-IL10: one VH-CH1-IL10 fusion and one light chain-IL10 fusion. VH-CH1-IL10: one IL-10 monomer is fused to the C-terminus of VH-CH1. Light chain-IL10: one IL-10 monomer is fused to the C-terminus of light chain (VL-CL-IL10). (9) F(ab′) 2-IL10: two VH-CH1-hinge-IL10 fusion and two light chains. VH-CH1-hinge-IL10 fusion: one IL-10 monomer is fused to the C-terminus of hinge of Fab heavy chain (VH1-CH1). (10) scFv-Fc-IL10: scFv is fused to the N-terminus of the human IgG hinge-CH2/CH3 domain to an IL-10 monomer. For example, the exemplary scFv-Fc-IL10 fusion protein is shown in SEQ ID NO: 34. In another embodiment, the exemplary scFv-IL10-IL10 fusion protein is shown in SEQ ID NO: 35.

An aspect of the invention provides a polynucleotide or vector encoding the fusion of the IL-10 and the LAG-3 targeting moiety. In one embodiment, the polynucleotide is an RNA encoding the fusion of the IL-10 and the LAG-3 targeting moiety. In one embodiment, the fusion may be complexed with a lipid nanoparticle (LNP) for in vivo delivery. In some embodiments, the LNP may comprise one or more of cationic lipid, neutral lipid, cholesterol-based lipid, and PEG-modified lipid. Methods for delivery of polynucleotide or vector are well known in the art. See WO2014152774A1, which is incorporated herein by reference.

In some embodiments, the polynucleotide or vector are modified to enhance stability.

In some embodiments, the polynucleotide or vector are modified to include a modified nucleotide, a modified sugar backbone, a cap structure, a poly A tail, a 5′ and/or 3′ untranslated region. In some embodiments, the polynucleotide or vector are unmodified.

In some embodiments, the polynucleotide or vector are administered intravenously. In some embodiments, the polynucleotide or vector are administered intraperitoneally. In some embodiments, the polynucleotide or vector are administered subcutaneously. In some embodiments, the polynucleotide or vector are administered via pulmonary administration.

In another aspect, the present invention provides a method of producing a fusion of the IL-10 and the LAG-3 targeting moiety by administering to a cell a polynucleotide or vector encoding the fusion, and wherein the fusion is produced by the cell. In some embodiments, the cell is a mammalian cell. In some embodiments, the cell is a human cell. In some embodiments, the cell is a cultured cell. In some embodiments, the cell is a cell within a living organism. In some embodiments, the fusion is expressed intracellularly. In some embodiments, the fusion is secreted by the cell.

In yet another aspect, the present invention provides compositions including polynucleotide or vector encoding the fusion of the IL-10 and the LAG-3 targeting moiety, wherein the polynucleotide or vector are encapsulated in one or more LNPs, e.g. liposomes.

The present invention provides, among other things, methods and compositions for delivering a fusion of the IL-10 and the LAG-3 targeting moiety in vivo based on mRNA delivery technology. In some embodiments, the present invention provides a method of delivery a fusion of the IL-10 and the LAG-3 targeting moiety by administering to a subject in need of delivery mRNAs encoding the fusion of the IL-10 and the LAG-3 targeting moiety. mRNAs may be delivered as packaged particles (e.g., encapsulated in liposomes or polymer based vehicles) or unpackaged (i.e., naked). mRNA encoded fusions may be expressed locally (e.g., in a tissue specific manner) or systematically in the subject.

According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags.

In some embodiments, a suitable delivery vehicle is formulated as a lipid nanoparticle (LNP). As used herein, the phrase “lipid nanoparticle” refers to a delivery vehicle comprising one or more lipids (e.g., cationic lipids, non-cationic lipids, cholesterol-based lipids, and PEG-modified lipids). The contemplated lipid nanoparticles may be prepared by including multi-component lipid mixtures of varying ratios employing one or more cationic lipids, non-cationic lipids, cholesterol-based lipids, and PEG-modified lipids. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides).

### III. Pharmaceutical Compositions and Formulations of the Combination

The present disclosure also provides pharmaceutical compositions and pharmaceutical formulations comprising the combination of the LAG-3 targeting moiety, e.g. an anti-LAG-3 antibody or a fragment thereof, and the IL-10. In some embodiments, the present disclosure provides a pharmaceutical formulation comprising the combination or the fusion of the LAG-3 targeting moiety, e.g. an anti-LAG-3 antibody or a fragment thereof, and the IL-10 as described herein and a pharmaceutically acceptable carrier. Such pharmaceutical formulations can be prepared by mixing the combination or the fusion protein thereof, having the desired degree of purity, with one or more pharmaceutically acceptable carriers. Typically, such formulations can be prepared as an aqueous solution (see e.g., U.S. Pat. No. 6,171,586, and WO2006/044908) or as a lyophilized formulation (see e.g., U.S. Pat. No. 6,267,958).

Pharmaceutically acceptable carriers are generally nontoxic to recipients at the dosages and concentrations employed. A wide range of such pharmaceutically acceptable carriers are well-known in the art (see e.g., Remington's Pharmaceutical Sciences 16th edition, Osol, A. Ed. (1980)). Exemplary pharmaceutically acceptable carriers useful in the formulations of the present disclosure can include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Pharmaceutically acceptable carriers useful in the formulations of the present disclosure can also include interstitial drug dispersion agents, such as soluble neutral-active hyaluronidase glycoproteins (sHASEGP) (see e.g., US Pat. Publ. Nos. 2005/0260186 and 2006/0104968), such as human soluble PH-20 hyaluronidase glycoproteins (e.g., rHuPH20 or HYLENEX®, Baxter International, Inc.).

It is also contemplated that the formulations disclosed herein may contain active ingredients in addition to the fusion protein thereof, as necessary for the particular indication being treated in the subject to whom the formulation is administered. Preferably, any additional active ingredient has activity complementary to that of the fusion protein thereof activity and the activities do not adversely affect each other.

In some embodiments, the formulation can be a sustained-release preparation of the active ingredients. Suitable examples of sustained-release preparations include semipermeable matrices of solid hydrophobic polymers containing the antibody, which matrices are in the form of shaped articles, e.g. films, or microcapsules.

Typically, the formulations of the present disclosure to be administered to a subject are sterile. Sterile formulations may be readily prepared using well-known techniques, e.g., by filtration through sterile filtration membranes.

In some embodiments of the methods of treatment of the present disclosure, the fusion protein thereof or pharmaceutical formulation comprising the combination or the fusion protein thereof is administered to a subject by any mode of administration that delivers the agent systemically, or to a desired target tissue. Systemic administration generally refers to any mode of administration of the antibody into a subject at a site other than directly into the desired target site, tissue, or organ, such that the antibody or formulation thereof enters the subject's circulatory system and, thus, is subject to metabolism and other like processes.

Accordingly, modes of administration useful in the methods of treatment of the present disclosure can include, but are not limited to, injection, infusion, instillation, and inhalation. Administration by injection can include intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion.

In some embodiments, a pharmaceutical formulation of the combination or the fusion protein thereof is formulated such that the LAG-3 targeting moiety and the IL-10 is protected from inactivation in the gut. Accordingly, the method of treatments can comprise oral administration of the formulation.

In some embodiments, use of the compositions or formulations comprising the combination or the fusion protein thereof of the present disclosure as a medicament are also provided. Additionally, in some embodiments, the present disclosure also provides for the use of a composition or a formulation comprising the combination or the fusion protein thereof in the manufacture or preparation of a medicament, particularly a medicament for treating, preventing or inhibiting disease. In a further embodiment, the medicament is for use in a method for treating, preventing or inhibiting a disease comprising administering to an individual having a disease an effective amount of the medicament. In certain embodiments, the medicament further comprises an effective amount of at least one additional therapeutic agent, or treatment.

The appropriate dosage of the combination or the fusion protein thereof contained in the compositions and formulations of the present disclosure (when used alone or in combination with one or more other additional therapeutic agents) will depend on the specific disease or condition being treated, the severity and course of the disease, whether the antibody is administered for preventive or therapeutic purposes, the previous therapy administered to the patient, the patient's clinical history and response to the antibody, and the discretion of the attending physician. The fusion protein thereof included in the compositions and formulations described herein, can be suitably administered to the patient at one time, or over a series of treatments. Various dosing schedules including but not limited to single or multiple administrations over various time-points, bolus administration, and pulse infusion are contemplated herein.

Depending on the type and severity of the disease, about 1 μg/kg to 15 mg/kg of fusion protein thereof in a formulation of the present disclosure is an initial candidate dosage for administration to a human subject, whether, for example, by one or more separate administrations, or by continuous infusion. Generally, the administered dosage of the combination or the fusion protein would be in the range from about 0.001 mg/kg to about 10 mg/kg. In some embodiments, one or more doses of about 0.5 mg/kg, 2.0 mg/kg, 4.0 mg/kg or 10 mg/kg (or any combination thereof) may be administered to a patient.

Dosage administration can be maintained over several days or longer, depending on the condition of the subject, for example, administration can continue until the disease is sufficiently treated, as determined by methods known in the art. In some embodiments, an initial higher loading dose may be administered, followed by one or more lower doses. However, other dosage regimens may be useful. The progress of the therapeutic effect of dosage administration can be monitored by conventional techniques and assays.

Accordingly, in some embodiments of the methods of the present disclosure, the administration of the fusion protein thereof comprises a daily dosage from about 1 mg/kg to about 100 mg/kg. In some embodiments, the dosage of fusion protein thereof comprises a daily dosage of at least about 0.01 mg/kg, at least about 0.1 mg/kg, at least about 1 mg/kg, at least about 10 mg/kg, or at least about 20 mg/kg.

### SPECIFIC EMBODIMENTS

[Embodiment 1] A combination of LAG-3 targeting moiety and IL-10.

[Embodiment 2] The combination of Embodiment 1, wherein the LAG-3 targeting moiety blocks LAG-3 from binding to ligands.

[Embodiment 3] The combination of any one of the precedent Embodiments, wherein the ligands comprise MHC class II, FGL-1, Gal-3 or lymph node sinusoidal endothelial cell C-type lectin (LSECtin).

[Embodiment 4] The combination of any one of the precedent Embodiments, wherein the LAG-3 targeting moiety is operably-linked or fused to the IL-10 via a linker.

[Embodiment 5] The combination of any one of the precedent Embodiments, wherein the linker comprises any one of the amino acid sequence of SEQ ID NO: 11-17.

[Embodiment 6] The combination of any one of the precedent Embodiments, wherein a fusion of the LAG-3 targeting moiety and IL-10 is selected from the group consisting of antibody-IL10; scFv-IL10; scFv-IL10-IL10; VH-IL10-IL10-VL; IL10-scFv-IL10; ScFv-CH1-IL10; IL10-scFv; Fab-IL10; F(ab′)₂-IL10 and scFv-Fc-IL10.

[Embodiment 7] The combination of any one of the precedent Embodiments, wherein the IL-10 is fused to the heavy chain of the antibody in the antibody-IL-10.

[Embodiment 8] The combination of any one of the precedent Embodiments, wherein the IL-10 is fused to N-terminal or C-terminal of the LAG-3 targeting moiety.

[Embodiment 9] The combination of any one of the precedent Embodiments, wherein:
a) the IL-10 is a naturally-occurring or engineered variant of IL-10 that retains its cytokine activity;
b) the IL-10 is a synthetically modified version of IL-10 that retains its cytokine activity;
c) the IL-10 comprises a substitution on amino acids, relative to amino acids of SEQ ID NO: 19: (1) R104Q; (2) any one of R107A, R107E, R107Q and R107D; or (3) a combination thereof.
d) the substitution comprises R104Q/R107A, R104Q/R107E, R104Q/R107Q or R104Q/R107D;
e) the IL-10 comprises an amino acid sequence having at least 80%, preferably at least 90%, and more preferably at least 95%, identity with SEQ ID NO: 19-29; and/or
f) the IL-10 is monomer or dimer.

[Embodiment 10] The combination of any one of the precedent Embodiments, wherein the LAG-3 targeting moiety is anti-LAG-3 antibody or a fragment thereof.

[Embodiment 11] The combination of any one of the precedent Embodiments, wherein the anti-LAG-3 antibody or a fragment thereof comprises a heavy chain (HC) fused via a linker to the IL-10 and a light chain (LC), wherein the HC and the LC comprises an amino acid sequence, respectively, having at least 80%, preferably 90%, or more preferably 95%, identity to:
a) SEQ ID NO: 1 and SEQ ID NO: 2;
b) SEQ ID NO: 3 and SEQ ID NO: 4;
c) SEQ ID NO: 5 and SEQ ID NO: 6;
d) SEQ ID NO: 32 and SEQ ID NO: 33.

[Embodiment 12] The combination of any one of the precedent Embodiments, wherein: (i) the antibody is a human, humanized, or chimeric antibody; (ii) the antibody is a full length antibody of class IgG, optionally, wherein the class IgG antibody has an isotype selected from IgG1, IgG2, IgG3, and IgG4; (iii) the antibody comprises an Fc region variant, optionally an Fc region variant that alters effector function and/or a variant that alters antibody half-life; (iv) the antibody is an antibody fragment, optionally selected from the group consisting of F(ab′)₂, Fab′, Fab, Fv, single domain antibody (VHH), and scFv; or (v) the antibody is a multi-specific antibody, optionally a bispecific antibody.

[Embodiment 13] The combination of any one of the precedent Embodiments, wherein the LAG-3 antibody or a fragment thereof comprises Relatlimab, Ieramilimab or Favezelimab.

[Embodiment 14] A polynucleotide or vector encoding the fusion of the IL-10 and the LAG-3 targeting moiety of any one of the precedent Embodiments.

[Embodiment 15] The polynucleotide or vector of any one of the precedent Embodiments, wherein the polynucleotide is an RNA encoding the fusion of the IL-10 and the LAG-3 targeting moiety.

[Embodiment 16] The polynucleotide or vector of any one of the precedent Embodiments, further comprising a lipid nanoparticle (LNP) complexed with the fusion.

[Embodiment 17] A host cell comprising the polynucleotide or vector of any one of the precedent Embodiments; optionally wherein the host cell is selected from a group consisting of Chinese hamster ovary (CHO) cell, a myeloma cell comprising Y0, NS0 or Sp2/0, a monkey kidney cell comprising COS-7, a human embryonic kidney line comprising 293, a baby hamster kidney cell (BHK), a mouse Sertoli cell comprising TM4, an African green monkey kidney cell comprising VERO-76, a human cervical carcinoma cell (HELA), a canine kidney cell, a human lung cell comprising W138, a human liver cell comprising HepG2, a mouse mammary tumor cell, a TRI cell, a Medical Research Council 5 (MRC 5) cell, a FS4 cell, neutrophils, eosinophils, basophils, mast cells, monocytes, macrophages, dendritic cells, and lymphocytes including natural killer cells, B cells and T cells.

[Embodiment 18] A method of producing the fusion of the IL-10 and the LAG-3 targeting moiety comprising culturing the host cell of any one of the precedent Embodiments.

[Embodiment 19] A pharmaceutical composition comprising the combination of any one of the precedent Embodiments, and a pharmaceutically acceptable carrier, diluent or excipient.

[Embodiment 20] Use of the combination of any one of the precedent Embodiments for the manufacture of a medicament for treating cancers.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Expression and Purification of Recombinant Proteins

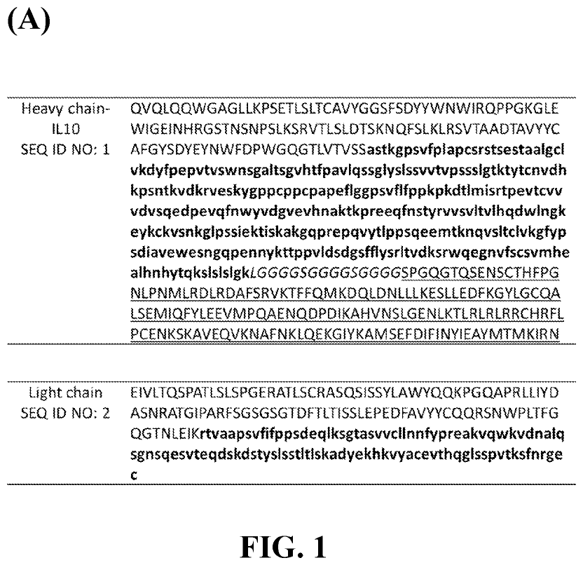

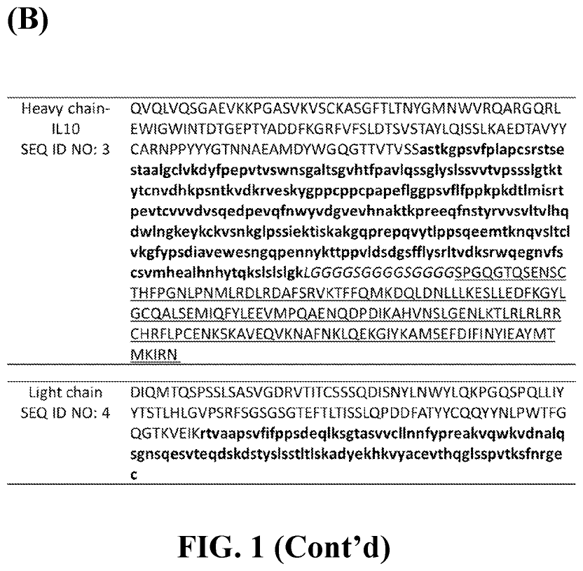

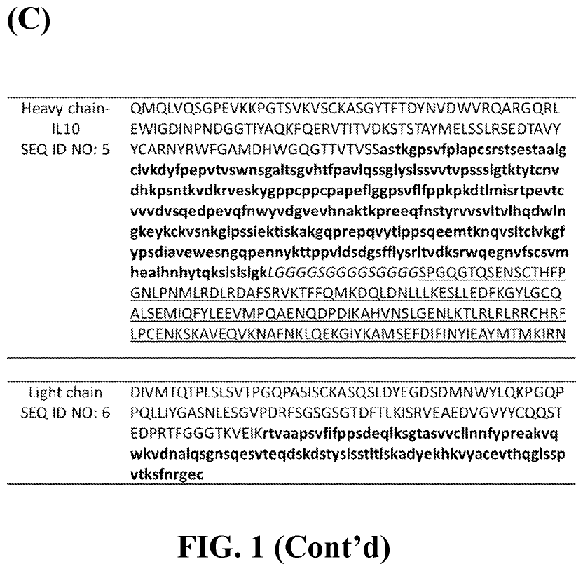

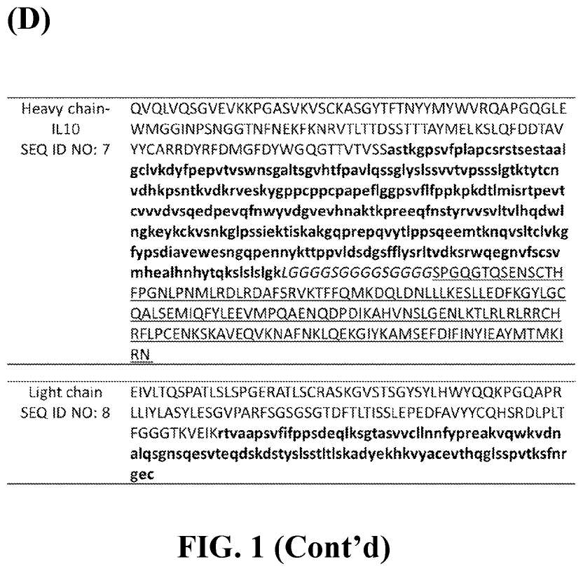

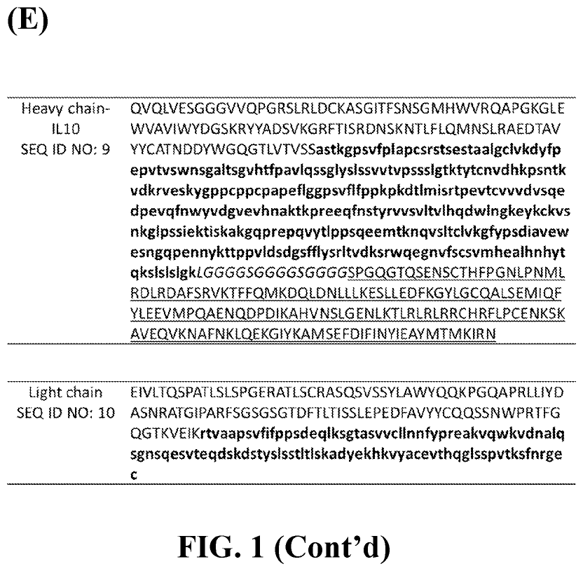

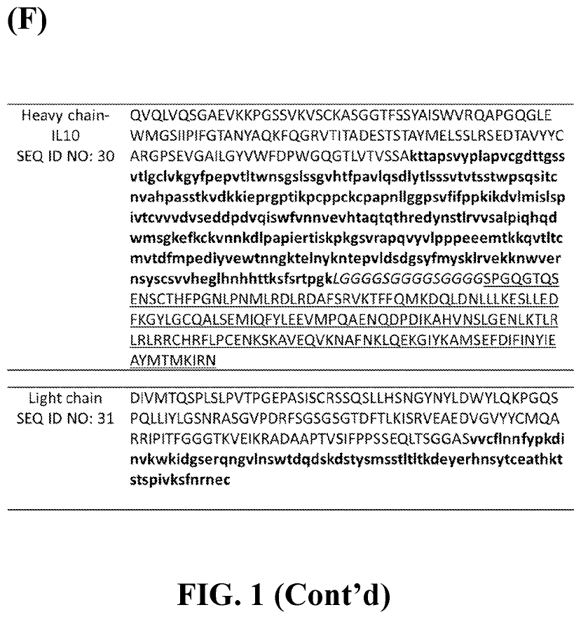

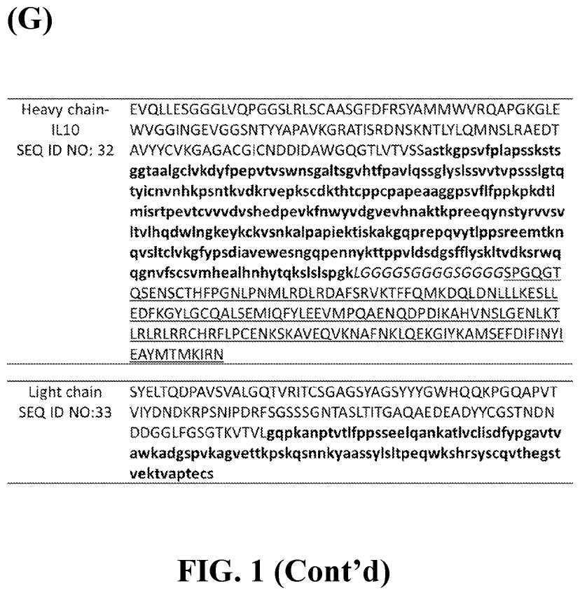

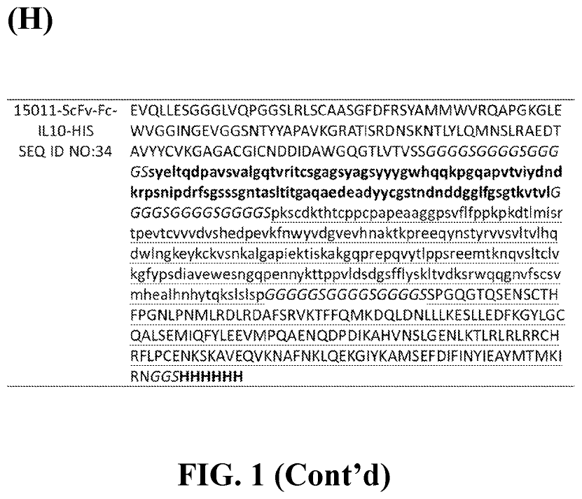

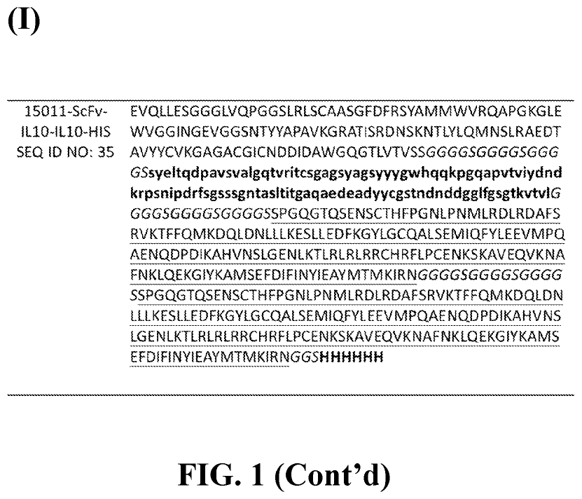

Plasmids coding for anti-PD-1 antibodies (Nivolumab and Pembrolizumab), anti-LAG-3 antibodies (Relatlimab, Ieramilimab and Favezelimab and 15011), anti-TIGIT antibody Clone 13 or the anti-LAG-3 antibody-IL10 fusion proteins (SEQ ID NO: 1-6 and 30-33, as shown in FIG. 1) were constructed by standard gene synthesis, followed by subcloning into a mammalian expression vector. The details of Relatlimab (BMS-986016 developed by Bristol-Myers Squibb), Ieramilimab (LAG525 developed by Novartis) and Favezelimab (MK-4280 developed by Merck) could be found in WO2014008218, US2015/0259420. The details of 15011 and Clone 13 could be found in WO2020041541 and US20200407444, respectively. Both 15011 and Clone 13 are human and mouse cross-reactive. The details of IL-10-Fc and anti-CSF-1R-IL10 fusion protein could be found in WO2023/060165. ExpiCHO cells were transfected with the constructed expression vectors according to the manual provided by the manufacturer (ThermoFisher, ExpiCHO™ Expression System Kit A29133). After culturing for 7-8 days, supernatant of transient expression products was collected and recombinant proteins were purified using protein A chromatography. Recombinant proteins were further purified to remove aggregates by gel filtration chromatography on a Superdex 200 Increase 10/300 GL column (GE Healthcare) with PBS.

### Example 2: Combination Treatment of IL-10 and Anti-LAG3 Antibody

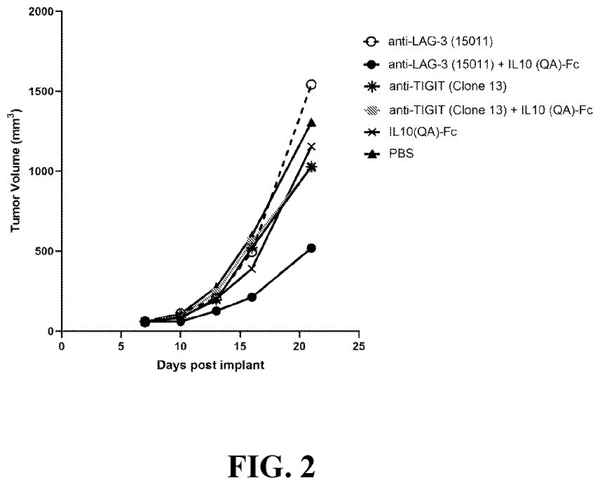

The anti-tumor activity of IL-10 R104Q/R107A (IL10 (QA))-Fc, anti-LAG3 antibody and of their combination was assessed in BALB/c mice bearing subcutaneously CT26-colorectal cell carcinoma. IL-10 (QA)-Fc exhibits similar anti-tumor activity and better developability and yield when compared to wild-type IL-10 and hence was used in this study. CT26 cells were inoculated subcutaneously into the upper back of BALB/c mice on day 0. When the subcutaneously transplanted tumor volume reached about 50-100 mm³, mice were randomly divided into control and treatment groups. Mice were treated with intraperitoneal injection of PBS, IL-10 (QA)-Fc (3 mg/kg), 15011 (anti-LAG3 antibody; 5 mg/kg), Clone 13 (anti-TIGIT antibody; 5 mg/kg), combination of 15011 and IL-10 (QA)-Fc (5 mg/kg and 3 mg/kg), or combination of Clone 13 and IL-10 (QA)-Fc (5 mg/kg and 3 mg/kg), administered on days 7, 11, 14, 17. As shown in FIG. 2, anti-LAG3 antibody 15011 alone did not inhibit tumor growth as expected and IL-10 (QA)-Fc alone did not show anti-tumor activity at the dose used in this study. In contrast, a significant anti-tumor activity for combination of IL-10 (QA)-Fc and 15011 was observed in vivo. Anti-TIGIT antibody Clone 13 showed no anti-tumor activity as a single agent or in combination with of IL-10 (QA)-Fc.

### Example 3: Effects of Fusion of IL-10 to Anti-LAG3 Antibody

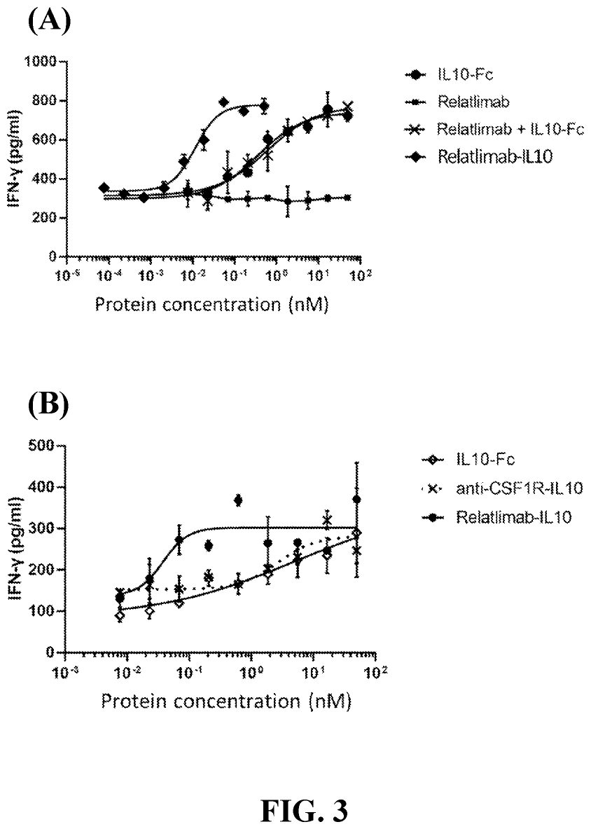

PBMCs from healthy adult donors were isolated from whole blood using Ficoll-Paque density gradient centrifugation. CD8 T cells were then purified by CD8 MicroBeads (Miltenyi Biotec #130-045-201). Purified CD8 T cells were activated by T cell TransAct (Miltenyi Biotec #130-111-160) in AIM-V culture medium for 3 days in humidified 37° C. incubator. After 3 days of culture, CD8 T cells were collected and washed in PBS three times. CD8 T cells were seeded at 1×10⁵ cells/well into a flat-bottom 96 well microplate in 100 mL of AIM-V culture medium. Relatlimab alone, Relatlimab combined with IL-10-Fc or Relatlimab fused with IL-10 (Relatlimab-IL10) was serially diluted and added to CD8 T cells for another 3 days. Then, 1 μg/ml OKT3 (Biolegend #317326) was added to CD8 T cells for 4 hours. CD8 T cell activation was monitored by determine the secreted IFN-γ in supernatants using ELISA (Biolegend #430101 and R&D #DY2906-05). As shown in FIG. 3A, IL-10-Fc induced IFN-γ secretion by CD8 T cells in a dose-dependent manner. LAG-3 antibody, Relatlimab, did not induce IFN-γ secretion. Combination of IL-10-Fc with Relatlimab induced a similar level of IFN-γ secretion level as seen with IL-10-Fc alone. Surprisingly, Relatlimab-IL10 fusion was markedly superior to other groups in its ability to induce IFN-γ secretion by CD8 T cells (FIG. 3A). The CD8 activation activity of Relatlimab-IL10 was also compared to an anti-CSF1R-IL10 fusion protein and IL-10-Fc which both do not target CD8 T cells (FIG. 3B). Relatlimab-IL10 induced significantly more IFN-γ secretion than anti-CSF-1R-IL10 and IL-10-Fc.

### Example 4: Superior CD8 Activation Effects of Anti-LAG3 Antibody-IL10 when Compared to Anti-PD-1-IL-10

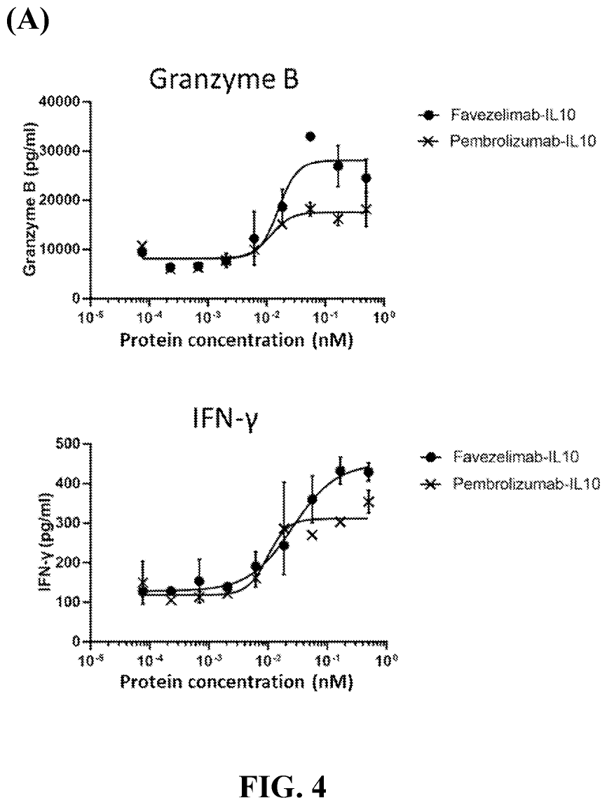

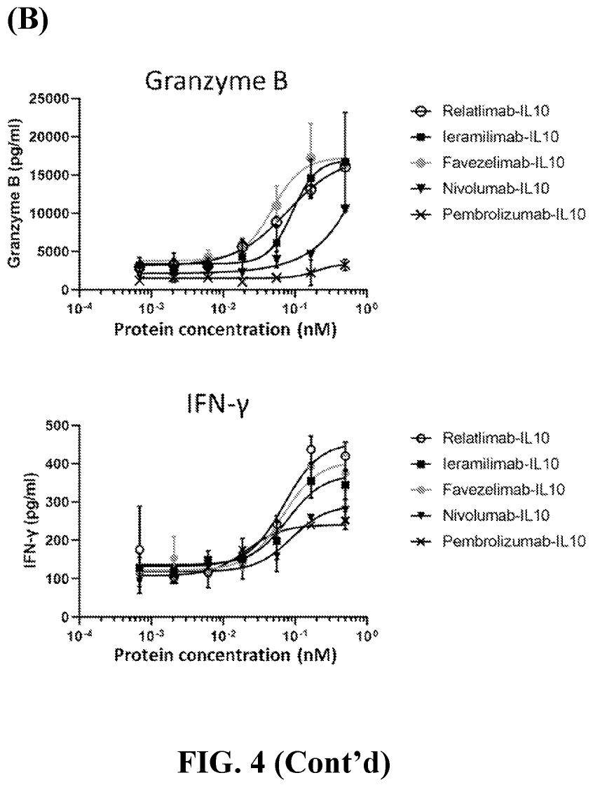

PBMCs from healthy adult donors were isolated from whole blood using Ficoll-Paque density gradient centrifugation. CD8 T cells were then purified by CD8 MicroBeads (Miltenyi Biotec #130-045-201). Purified CD8 T cells were activated by T cell TransAct (Miltenyi Biotec #130-111-160) in AIM-V culture medium for 3 days in humidified 37° C. incubator. After 3 days of culture, CD8 T cells were collected and washed in PBS three times. CD8 T cells were seeded at 1×10⁵ cells/well into a flat-bottom 96 well microplate in 100 ml of AIM-V culture medium. Purified recombinant fusion proteins anti-LAG3 antibody-IL10 and anti-PD-1-IL10 were serially-diluted and added to CD8 T cells for another 3 days. Then, 1 μg/ml OKT3 (Biolegend #317326) was added to CD8 T cells for 4 hours. Secreted IFN-γ and granzyme b were then determined by ELISA (Biolegend #430101 and R&D #DY2906-05). As shown in FIG. 4(A) and 4(B), surprisingly, the potency to stimulate CD8 T cells by all three different anti-LAG3 antibody-IL10 fusion proteins (Relatlimab-IL10, Ieramilimab-IL10, and Favezelimab-IL10) was higher than that of anti-PD-1-IL10 fusion proteins tested (Pembrolizumab-IL10 and Nivolumab-IL10).

### Example 5: Anti-LAG3 Antibody-IL10 Fusion Protein Secreted from Cells in Culture Supernatant could Enhance CD8 T Cell Activation

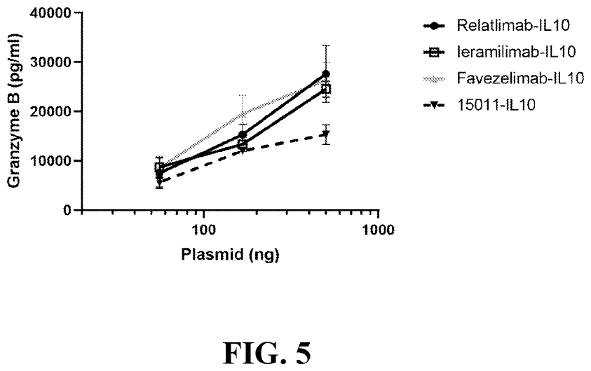

Polynucleotide delivery is an attractive strategy to achieve in vivo expression of protein drugs. The biological activity of culture supernatant containing anti-LAG3 antibody-IL10 fusion protein secreted by transduced cells was investigated. HeLa cells were transfected with increasing amounts of plasmids encoding various anti-LAG3 antibody-IL10 fusion proteins (Relatlimab-IL10, Ieramilimab-IL10 and favezelimab-IL10, and 15011-IL10) following lipofectamine 2000 manufacturer's protocol. After 72 hours, cell culture supernatants were taken and added to CD8 T cell activation assay as described in example 2. Secreted granzyme B from CD8 T cells were determined by ELISA. As shown in FIG. 5, anti-LAG3 antibody-IL10 fusion proteins secreted by human tumor cells in culture supernatant exhibited CD8 activation activity in a dose dependent manner.

### Example 6: Anti-Tumor Efficacy of Anti-LAG3 Antibody-IL10 mRNA

To prepare mRNA for in vivo study, the DNA comprising a codon-optimized nucleotide sequence (SEQ ID NO: 36 and 37) encoding 15011-human IL-10 polypeptide (SEQ ID NO: 32 and 33) and a negative control luciferase polypeptide were cloned into the vector provided by Takara IVTpro mRNA Synthesis System (Cat. #6141). mRNA were synthesized with full substitution of UTP by pseudouridine-5′-triphosphate (TriLink, USA) from the corresponding linearized plasmids by in vitro transcription using Takara IVTpro mRNA Synthesis System (Cat. #6141). Cap 1 structure was added using CleanCap Reagent AG (3′ OMe) (TriLink: N7413). LNP-mRNA was prepared by mixing lipid materials dissolved in ethanol and mRNA diluted in 50 mM sodium acetate buffer, pH=3.0. The molar ratio of LNPs was (SM-102:DSPC:cholesterol:DMPE-PEG2000)=50:10:38.5:1.5. LNPs-mRNA were prepared by rapidly mixing the aqueous and ethanol phases using vortex mixing. The mRNA encapsulation efficiency was determined by the RiboGreen assay (Invitrogen, USA). Heavy chain-IL10 and light chain of 15011-IL10 were encoded on individual mRNAs and formulated with LNP separately and then the two mRNA-LNP were mixed together in a 2:1 mRNA molar ratio.

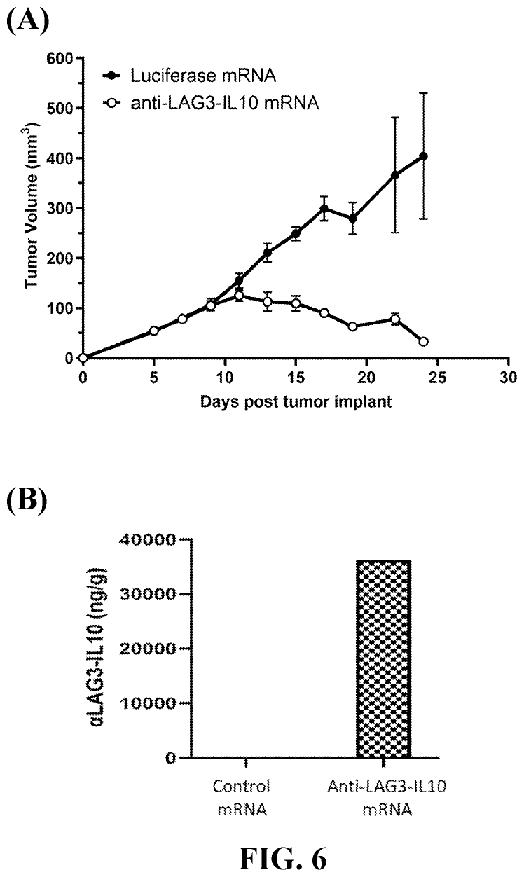

MC38 cells were inoculated subcutaneously on the upper back of 6-8 weeks old female BALB/cJ mice. When the subcutaneously transplanted tumor volume reached about 50-100 mm³, mice were randomly divided into control and treatment groups, and intratumoral administration of mRNA-LNP was performed, and the dosage of mRNA group was 12 μg per mouse, once every 3 days for the first week and then 6 μg per mouse, once every 3 days for the second week (a total of four times). As shown in FIG. 6(A), intra-tumoral injection of mRNA encoding 15011-IL10 fusion protein markedly suppressed the tumor growth. Tumor tissues were harvested 24 hours after administration, homogenized in Tris/HCl buffer and centrifuged. The clear supernatant was subsequently analyzed for the 15011-IL10 fusion protein expression by injected mRNA using ELISA. Briefly, Nunc MaxiSorp™ flat-bottom plates (Invitrogen) were coated with anti-IL10 Ab (Biolegend 506802) diluted to 1 μg/mL (100 μl/well). The plates were washed with PBST and blocked with 1% BSA. After washing with PBST once, dilutions of the tumor lysate samples were added, along with a reference standard. Stocks of known concentration for 15011-IL10 were used to make the standard curve. After 1 hour at room temp, the plate was washed 3 times and then anti-human Lambda HRP (BETHYL A80116P) diluted to 0.1 μg/mL was added. Following an additional 3 washes, the plates were incubated in the dark with room-temperature TMB (Thermo Scientific). The reaction was stopped with 100 μL/well 2 N HCL. Absorbance was assessed at 450 nm and 650 nm. As shown in FIG. 6(B), 15011-IL10 protein expressed by mRNA could be detected in tumor lysates.

### Example 7: In Vivo Efficacy of 15011-IL10 mRNA Compared to Combination of 15011 mRNA and IL10 mRNA

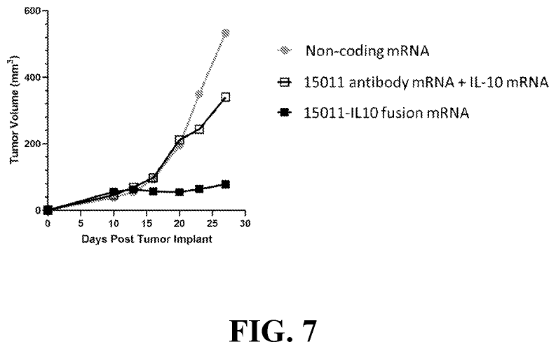

CT26 cells were inoculated subcutaneously into the upper back of BALB/c mice. When the subcutaneously transplanted tumor volume reached about 50-100 mm³, mice were randomly divided into control and treatment groups, and intratumoral administration was performed. Mice were treated once a week for 2 weeks with fusion molecule 15011-IL10 mRNA-LNP (4 μg), non-coding mRNA-LNP (12 μg) or combinations of 15011 antibody mRNA-LNP (3 μg) and human IL10 mRNA-LNP (1 μg). Approximately, same mRNA copy number of test article was used in this study to compare the anti-tumor activity between fusion and combination. As shown in FIG. 7, combination of mRNA encoding 15011 antibody alone and IL10 alone exhibited anti-tumor activity. Surprisingly, mRNA encoding 15011-IL10 fusion proteins was more potent than combination of two mRNAs encoding 15011 and IL-10 respectively.

### Example 8: Different Formats of Anti-LAG3 Antibody-IL10 Fusion

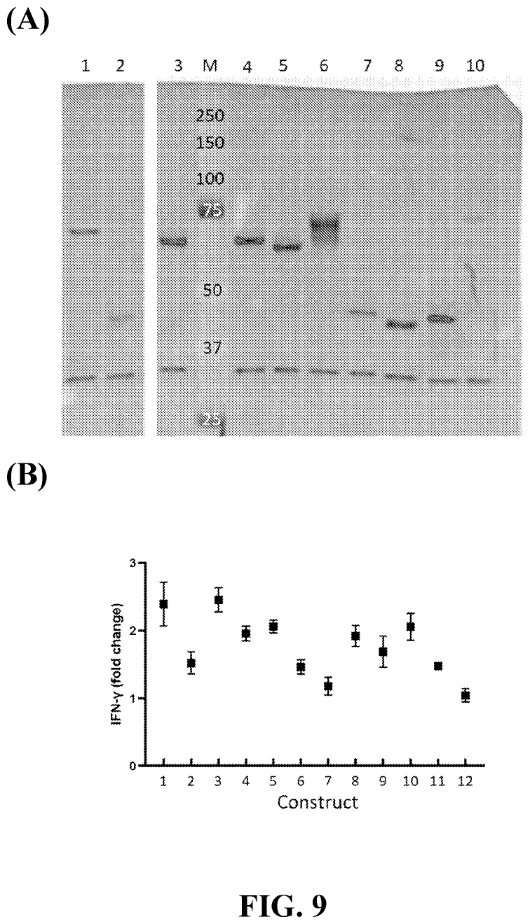

To investigate different fusion formats to see how expression and functional levels were affected, ten formats comprise one or two IL-10 fused with IgG, scFv, scFv-Fc, scFv-CH1 or Fab of an anti-human LAG3 antibody hybridoma clone 4 were constructed into pcDNA3.4 (FIG. 8). IL-10 and scFv of the anti-human LAG3 antibody clone 4 were also constructed into pcDNA3.4 as the controls. HeLa cells were seeded at 2×10⁴ cells/well in 96-well plates. Transfection using Lipofectamine 2000 was performed according to the manufacturer's protocol. Culture supernatant was collected 72 hours post-transfection for either CD8 activation assay or protein expression study. For protein expression study, culture supernatants from each construct transfection were loaded on BisTris 4%-12% polyacrylamide gel and blotted on PVDF membranes. IL-10 containing protein fragment were detected by anti-IL10 antibody (GeneTex, GTX-130513). As shown in FIG. 9(A), all formats of fusion proteins could be expressed. CD8 assay was performed as described in example 1. IFN-γ level released in culture supernatant form various IL-10 fusion constructs was determined by ELISA and normalized against scFv which does not increase IFN-γ secretion in this assay. As shown in FIG. 9(B), culture supernatants from various IL-10 fusion constructs showed enhanced CD8 activation. These data demonstrated that anti-LAG3 antibody-IL10 fusion proteins could be produced and released in cell culture supernatant and the released proteins exhibited CD8 activation activity.

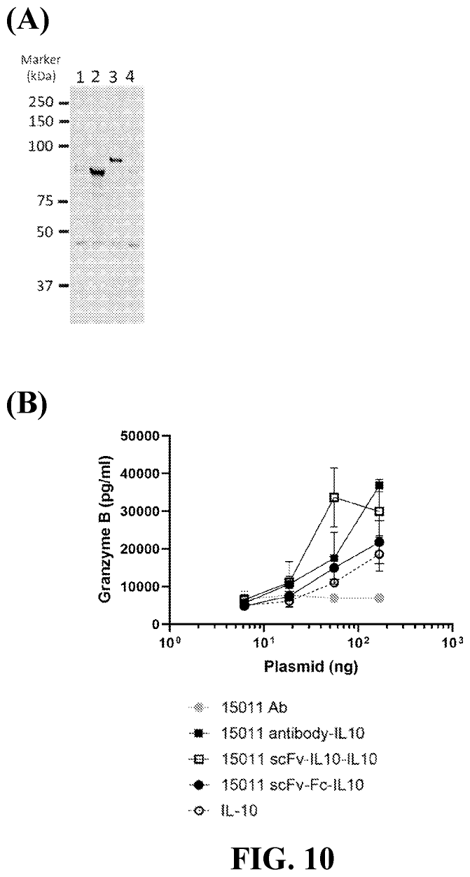

The activity of three fusion formats was further confirmed using another anti-LAG3 antibody, 15011. Expression vector for 15011 antibody-IL10 (15011-IL10) (SEQ ID NO: 32 and 33), 15011 scFv-IL10-IL10-His (SEQ ID NO: 35) and 15011 scFv-Fc-IL10-His (SEQ ID NO: 34) were also constructed using pcDNA3.4 (FIGS. 1G-1I). Expression and CD8 activation activity were performed as described. As shown in FIG. 10, three formats could be expressed and had good CD8 activation activity.

Notwithstanding the appended claims, the disclosure set forth herein is also defined by the following clauses, which may be beneficial alone or in combination, with one or more other causes or embodiments. Without limiting the foregoing description, certain non-limiting clauses of the disclosure numbered as below are provided, wherein each of the individually numbered clauses may be used or combined with any of the preceding or following clauses. Thus, this is intended to provide support for all such combinations and is not necessarily limited to specific combinations explicitly provided below:

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

### REFERENCES

1. Rowbottom, A. W., Lepper, M. A., Garland, R. J., Cox, C. V., and Corley, E. G. (1999). Interleukin-10-induced CD8 cell proliferation. Immunology 98, 80-89. 10.1046/j.1365-2567.1999.00828.x.
2. Fujii, S., Shimizu, K., Shimizu, T., and Lotze, M. T. (2001). Interleukin-10 promotes the maintenance of antitumor CD8 (+) T-cell effector function in situ. Blood 98, 2143-2151. 10.1182/blood.v98.7.2143.
3. Emmerich, J., Mumm, J. B., Chan, I. H., LaFace, D., Truong, H., McClanahan, T., Gorman, D. M., and Oft, M. (2012). IL-10 directly activates and expands tumor-resident CD8 (+) T cells without de novo infiltration from secondary lymphoid organs. Cancer Res 72, 3570-3581. 10.1158/0008-5472.CAN-12-0721.
4. Guo, Y., Xie, Y.-Q., Gao, M., Zhao, Y., Franco, F., Wenes, M., Siddiqui, I., Bevilacqua, A., Wang, H., Yang, H., et al. (2021). Metabolic reprogramming of terminally exhausted CD8+ T cells by IL-10 enhances anti-tumor immunity. Nat Immunol 22, 746-756. 10.1038/s41590-021-00940-2.
5. Naing, A., Infante, J. R., Papadopoulos, K. P., Chan, I. H., Shen, C., Ratti, N. P., Rojo, B., Autio, K. A., Wong, D. J., Patel, M. R., et al. (2018). PEGylated IL-10 (Pegilodecakin) Induces Systemic Immune Activation, CD8+ T Cell Invigoration and Polyclonal T Cell Expansion in Cancer Patients. Cancer Cell 34, 775-791.e3. 10.1016/j.ccell.2018.10.007.

What is claimed is:

1. A combination of LAG-3 targeting moiety and IL-10.

2. The combination of claim 1, wherein the LAG-3 targeting moiety blocks LAG-3 from binding to ligands.

3. The combination of claim 2, wherein the ligands comprise MHC class II, FGL-1, Gal-3 or lymph node sinusoidal endothelial cell C-type lectin (LSECtin).

4. The combination of claim 1, wherein the LAG-3 targeting moiety is operably-linked or fused to the IL-10 via a linker.

5. The combination of claim 4, wherein the linker comprises any one of the amino acid sequence of SEQ ID NO: 11-17.

6. The combination of claim 4, wherein a fusion of the LAG-3 targeting moiety and IL-10 is selected from the group consisting of Antibody-IL10; scFv-IL10; scFv-IL10-IL10; VH-IL10-IL10-VL; IL10-scFv-IL10; ScFv-CH1-IL10; IL10-scFv; Fab-IL10; F(ab′) 2-IL10 and scFv-Fc-IL10.

7. The combination of claim 6, wherein the IL-10 is fused to the heavy chain of the antibody in the antibody-IL10.

8. The combination of claim 1, wherein the IL-10 is fused to N-terminal or C-terminal of the LAG-3 targeting moiety.

9. The combination of claim 1, wherein:
(a) the IL-10 is a naturally-occurring or engineered variant of IL-10 that retains its cytokine activity;
(b) the IL-10 is a synthetically modified version of IL-10 that retains its cytokine activity;
(c) the IL-10 comprises a substitution on amino acids, relative to amino acids of SEQ ID NO: 19: (1) R104Q; (2) any one of R107A, R107E, R107Q and R107D; or (3) a combination thereof.
(d) the substitution comprises R104Q/R107A, R104Q/R107E, R104Q/R107Q or R104Q/R107D;
(e) the IL-10 comprises an amino acid sequence having at least 80%, preferably at least 90%, and more preferably at least 95%, identity with SEQ ID NO: 19-29; and/or
(f) the IL-10 is monomer or dimer.

10. The combination of claim 1, wherein the LAG-3 targeting moiety is anti-LAG-3 antibody or a fragment thereof.

11. The combination of claim 10, wherein the anti-LAG-3 antibody or a fragment thereof comprises a heavy chain (HC) fused via a linker to the IL-10 and a light chain (LC), wherein the HC and the LC comprises an amino acid sequence, respectively, having at least 80%, preferably 90%, or more preferably 95%, identity to:
(a) SEQ ID NO: 1 and SEQ ID NO: 2;
(b) SEQ ID NO: 3 and SEQ ID NO: 4;
(c) SEQ ID NO: 5 and SEQ ID NO: 6; or
(d) SEQ ID NO: 32 and SEQ ID NO: 33.

12. The combination of claim 10, wherein: (i) the antibody is a human, humanized, or chimeric antibody; (ii) the antibody is a full length antibody of class IgG, optionally, wherein the class IgG antibody has an isotype selected from IgG1, IgG2, IgG3, and IgG4; (iii) the antibody comprises an Fc region variant, optionally an Fc region variant that alters effector function and/or a variant that alters antibody half-life; (iv) the antibody is an antibody fragment, optionally selected from the group consisting of F(ab′)₂, Fab′, Fab, Fv, single domain antibody (VHH), and scFv; or (v) the antibody is a multi-specific antibody, optionally a bispecific antibody.

13. The combination of claim 10, wherein the LAG-3 antibody or a fragment thereof comprises 15011, Relatlimab, Ieramilimab or Favezelimab.

14. A polynucleotide or vector encoding the fusion of the IL-10 and the LAG-3 targeting moiety of claim 8.

15. The polynucleotide or vector of claim 14, wherein the polynucleotide is an RNA encoding the fusion of the IL-10 and the LAG-3 targeting moiety.

16. The polynucleotide or vector of claim 15, further comprising a lipid nanoparticle (LNP) complexed with the fusion.

17. A host cell comprising the polynucleotide or vector of claim 14; optionally wherein the host cell is selected from a group consisting of Chinese hamster ovary (CHO) cell, a myeloma cell comprising Y0, NS0 or Sp2/0, a monkey kidney cell comprising COS-7, a human embryonic kidney line comprising 293, a baby hamster kidney cell (BHK), a mouse Sertoli cell comprising TM4, an African green monkey kidney cell comprising VERO-76, a human cervical carcinoma cell (HELA), a canine kidney cell, a human lung cell comprising W138, a human liver cell comprising HepG2, a mouse mammary tumor cell, a TRI cell, a Medical Research Council 5 (MRC 5) cell, a FS4 cell, neutrophils, eosinophils, basophils, mast cells, monocytes, macrophages, dendritic cells, and lymphocytes including natural killer cells, B cells and T cells.

18. A method of producing the fusion of the IL-10 and the LAG-3 targeting moiety comprising culturing the host cell of claim 17.

19. A pharmaceutical composition comprising a combination of any one of claims 1-13, and a pharmaceutically acceptable carrier, diluent or excipient.

20. Use of a combination of any one of claims 1-13 for the manufacture of a medicament for treating cancers.


SEQUENCE LISTING (37 sequences)

SEQ ID NO: 1 - protein, 622 aa, synthetic construct
  REGION 1..120: note = Variable domain
  REGION 121..447: note = Constant domain
  REGION 448..462: note = Linker
  REGION 463..622: note = IL-10
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 214 aa, synthetic construct
  REGION 1..107: note = Variable domain
  REGION 108..214: note = Constant domain
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 627 aa, synthetic construct
  REGION 453..467: note = Linker
  REGION 126..452: note = Constant domain
  REGION 1..125: note = Variable domain
  REGION 468..627: note = IL-10
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 4 - protein, 214 aa, synthetic construct
  REGION 1..107: note = Variable domain
  REGION 107..214: note = Constant domain
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 621 aa, synthetic construct
  REGION 462..621: note = IL-10
  REGION 1..119: note = Variable domain
  REGION 447..461: note = Linker
  REGION 120..446: note = Constant domain
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 6 - protein, 218 aa, synthetic construct
  REGION 1..111: note = Variable domain
  REGION 112..218: note = Constant domain
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 622 aa, synthetic construct
  REGION 463..622: note = IL-10
  REGION 1..120: note = Variable domain
  REGION 448..462: note = Linker
  REGION 121..447: note = Constant domain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 8 - protein, 218 aa, synthetic construct
  REGION 1..111: note = Variable domain
  REGION 112..218: note = Constant domain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 615 aa, synthetic construct
  REGION 456..612: note = IL-10
  REGION 1..113: note = Variable domain
  REGION 441..455: note = Linker
  REGION 114..440: note = Constant domain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 10 - protein, 214 aa, synthetic construct
  REGION 1..107: note = Variable domain
  REGION 108..214: note = Constant domain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
  REPEAT 1..5: note = 1-6 repeat(s)
GGGGS

SEQ ID NO: 12 - protein, 5 aa, synthetic construct
  REPEAT 1..5: note = 1-6 repeat(s)
SSSSG

SEQ ID NO: 13 - skipped

SEQ ID NO: 14 - protein, 5 aa, synthetic construct
  REPEAT 1..5: note = 1-6 repeat(s)
EAAAK

SEQ ID NO: 15 - skipped

SEQ ID NO: 16 - protein, 7 aa, synthetic construct
  VARIANT 7: note = X is G or S
ENLYFQX

SEQ ID NO: 17 - protein, 15 aa, synthetic construct
LGGGGSGGGGSGGGG

SEQ ID NO: 18 - protein, 178 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 19 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 20 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRRCHRFLPCENKSKAVEQVKNAFNKLQEKGIYKAMSEFDIFINYIEAYMTMKIRN

SEQ ID NO: 21 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRACHRFLPCENKSKAVEQVKNAFNKLQEKGIYKAMSEFDIFINYIEAYMTMKIRN

SEQ ID NO: 22 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRECHRFLPCENKSKAVEQVKNAFNKLQEKGIYKAMSEFDIFINYIEAYMTMKIRN

SEQ ID NO: 23 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRQCHRFLPCENKSKAVEQVKNAFNKLQEKGIYKAMSEFDIFINYIEAYMTMKIRN

SEQ ID NO: 24 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRDCHRFLPCENKSKAVEQVKNAFNKLQEKGIYKAMSEFDIFINYIEAYMTMKIRN

SEQ ID NO: 25 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSSAVEQVKNAFNKLQEKGIYKAMSEFDIFINYIEAYMTMKIRN

SEQ ID NO: 26 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRACHRFLPCENKSKAVEQVKNAFNKLQEKGIYKAMSEFDIFINYIEAYMTMKIRN

SEQ ID NO: 27 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRECHRFLPCENKSKAVEQVKNAFNKLQEKGIYKAMSEFDIFINYIEAYMTMKIRN

SEQ ID NO: 28 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRQCHRFLPCENKSKAVEQVKNAFNKLQEKGIYKAMSEFDIFINYIEAYMTMKIRN

SEQ ID NO: 29 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLQLRDCHRFLPCENKSKAVEQVKNAFNKLQEKGIYKAMSEFDIFINYIEAYMTMKIRN

SEQ ID NO: 30 - protein, 630 aa, synthetic construct
  REGION 1..126: note = Variable domain
  REGION 127..455: note = Constant domain
  REGION 456..470: note = Linker
  REGION 471..630: note = IL-10
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 31 - protein, 219 aa, synthetic construct
  REGION 137..219: note = Constant domain
  REGION 1..136: note = Variable domain
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 630 aa, synthetic construct
  REGION 1..125: note = Variable domain
  REGION 126..455: note = Constant domain
  REGION 456..470: note = Linker
  REGION 471..630: note = IL-10
EVQLLESGGGLVQPGGSLRLSCAASGFDFRSYAMMWVRQAPGKGLEWVGGINGEVGGSNTYYAPAVKGRATISRDNSKNTLYLQMNSLRAEDTAVYYCVKGAGACGICNDDIDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 33 - protein, 216 aa, synthetic construct
  REGION 1..110: note = Variable domain
  REGION 111..216: note = Constant domain
SYELTQDPAVSVALGQTVRITCSGAGSYAGSYYYGWHQQKPGQAPVTVIYDNDKRPSNIPDRFSGSSSGNTASLTITGAQAEDEADYYCGSTNDNDDGGLFGSGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 34 - protein, 679 aa, synthetic construct
  REGION 1..125: note = Heavy Chain Variable domain
  REGION 126..140: note = Linker
  REGION 141..250: note = Light Chain Variable domain
  REGION 251..265: note = Linker
  REGION 266..494: note = FC
  REGION 495..510: note = Linker
  REGION 511..670: note = IL-10
  REGION 671..673: note = Linker
  REGION 674..679: note = HIS tag
EVQLLESGGGLVQPGGSLRLSCAASGFDFRSYAMMWVRQAPGKGLEWVGGINGEVGGSNTYYAPAVKGRATISRDNSKNTLYLQMNSLRAEDTAVYYCVKGAGACGICNDDIDAWGQGTLVTVSSGGGGSGGGGSGGGGSSYELTQDPAVSVALGQTVRITCSGAGSYAGSYYYGWHQQKPGQAPVTVIYDNDKRPSNIPDRFSGSSSGNTASLTITGAQAEDEADYYCGSTNDNDDGGLFGSGTKVTVLGGGGSGGGGSGGGGSPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHH

SEQ ID NO: 35 - protein, 609 aa, synthetic construct
  REGION 1..125: note = Heavy Chain Variable domain
  REGION 126..140: note = Linker
  REGION 141..250: note = Light Chain Variable domain
  REGION 251..265: note = Linker
  REGION 266..425: note = IL-10
  REGION 426..440: note = Linker
  REGION 441..600: note = IL-10
  REGION 601..603: note = Linker
  REGION 604..609: note = HIS tag
EVQLLESGGGLVQPGGSLRLSCAASGFDFRSYAMMWVRQAPGKGLEWVGGINGEVGGSNTYYAPAVKGRATISRDNSKNTLYLQMNSLRAEDTAVYYCVKGAGACGICNDDIDAWGQGTLVTVSSGGGGSGGGGSGGGGSSYELTQDPAVSVALGQTVRITCSGAGSYAGSYYYGWHQQKPGQAPVTVIYDNDKRPSNIPDRFSGSSSGNTASLTITGAQAEDEADYYCGSTNDNDDGGLFGSGTKVTVLGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSHHHHHH

SEQ ID NO: 36 - DNA, 1944 bp, synthetic construct
  source 1..1944: mol_type = other DNA
  sig_peptide 1..198
atgggttggagcctcatcttgctcttccttgtcgctgttgctacgcgtgtgctgtctgaggttcagctgctggaatctggcggaggattggttcagcctggcggctctctgagactgtcttgtgccgcctctggcttcgacttccggtcctacgctatgatgtgggtccgacaggctcctggcaaaggcctggaatgggtcggaggcatcaatggcgaagtcggcggctccaacacctactatgcccctgctgtgaagggcagagccaccatctccagagacaactccaagaacaccctgtacctgcagatgaactccctgagagccgaggacaccgccgtgtactactgtgtgaaaggcgctggcgcctgcggcatctgcaacgatgatatcgatgcttggggccagggcaccctggtcacagttagttctgctagcacaaagggcccctctgtgtttcccctggctcctagcagcaagagcacatctggtggaacagccgctctgggctgcctggtcaaggattactttcccgagcctgtgaccgtgtcctggaatagcggagcactgacaagcggcgtgcacacctttccagccgtgctgcaaagcagcggcctgtactctctgagcagcgtggtcacagtgccaagctctagcctgggcacccagacctacatctgcaatgtgaaccacaagcctagcaacaccaaggtggacaagcgtgtggaacccaagagctgcgacaagacccacacctgtcctccatgtcctgctccagaagctgccggcggaccttccgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcctgctcctatcgagaaaaccatcagcaaggccaagggccagccaagagaaccccaggtgtacacactgcctccaagccgggaagagatgaccaagaaccaggtgtccctgacctgcctcgtgaagggcttttaccccagcgacattgccgtggaatgggagagcaatggccagcctgagaacaactacaagacaacccctcctgtgctggacagcgacggctcattcttcctgtacagcaagctgaccgtggacaagtccagatggcagcagggcaacgtgttcagctgctccgtgatgcacgaggccctgcacaaccactacacccagaagtccctgagcctgtctcctggcggcggcggaggatctggcggaggtggaagcggaggcggtggatctcctggacagggaacacagagcgagaacagctgcacccactttccaggcaacctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccctgacattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatcagaaactaa

SEQ ID NO: 37 - DNA, 711 bp, synthetic construct
  source 1..711: mol_type = other DNA
  sig_peptide 1..60
atgagagtgcccgctcagctgctgggactgctgttgttgtggttgcctggcgctcggtgctcctacgaactgacccaagatcctgccgtgtctgtggctctgggccagacagtgcggatcacatgttctggcgctggctcttacgccggctcctactactatggctggcaccagcagaagcctggacaggctcctgtgaccgtgatctacgacaacgacaagcggcccagcaacatccctgacagattctccggctcctccagcggcaataccgccagcttgacaattaccggcgctcaggctgaggacgaggccgattactattgcggctccaccaacgataacgacgacggcggcttgtttggctccggcaccaaagtgacagtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcctaa